SEQUENCE LISTING (9 sequences)

SEQ ID NO: 1 - DNA, 32 bp, Artificial Sequence
  S. aureus Forward Primer
cgctactagttgcttagtgttaactttagttg

SEQ ID NO: 2 - DNA, 28 bp, Artificial Sequence
  S. aureus Reverse Primer
tgcactatatactgttggatcttcagaa

SEQ ID NO: 3 - DNA, 33 bp, Artificial Sequence
  S. aureus Probe
tgcatcacaaacagataacggcgtaaatagaag

SEQ ID NO: 4 - DNA, 23 bp, Artificial Sequence
  S. enterica Forward Primer
ctcaccaggagattacaacatgg

SEQ ID NO: 5 - DNA, 23 bp, Artificial Sequence
  S. enterica Reverse Primer
agctcagaccaaaagtgaccatc

SEQ ID NO: 6 - DNA, 22 bp, Artificial Sequence
  S. enterica Probe
caccgacggcgagaccgacttt

SEQ ID NO: 7 - DNA, 18 bp, Artificial Sequence
  C. albicans Forward Probe
ctgtttgagcgtcgtttc

SEQ ID NO: 8 - DNA, 21 bp, Artificial Sequence
  C. albicans Reverse Primer
atgcttaagttcagcgggtag

SEQ ID NO: 9 - DNA, 24 bp, Artificial Sequence
  C. albicans Probe
ctgggtttggtgttgagcaatacg

(19) United States
(12) Patent Application Publication
(10) Pub. No.: US 2011/0281266 A1
(43) Pub. Date: Nov. 17, 2011

(54) Identification of Nucleic Acids

(75) Inventors: Nikolay V. Sergeev, Foster City, CA (US); Maxim G. Brevnov, Union City, CA (US); Manohar R. Furtado, San Ramon, CA (US)

(73) Assignee: LIFE TECHNOLOGIES CORPORATION, Carlsbad, CA (US)

(21) Appl. No.: 13/106,736

(22) Filed: May 12, 2011

Related U.S. Application Data
(60) Provisional application No. 61/334,803, filed on May 14, 2010.

(51) Int. Cl. C12Q 1/68

(52) U.S. Cl. 435/6.11

(57) ABSTRACT

This disclosure relates to methods for identifying target nucleic acids in a sample by detecting an amplified sequence corresponding to the target using a detectable probe and by monitoring its melting temperature (Tₘ).

### CROSS-REFERENCE TO RELATED APPLICATIONS

This application claims the benefit of priority under 35 U.S.C. §119(e) to U.S. Provisional Patent Application No. 61/334,803, filed May 14, 2010, which is herein incorporated by reference in its entirety.

### FIELD

This disclosure relates to methods for identifying target nucleic acids in a sample by detecting an amplified sequence corresponding to the target using a detectable probe and by monitoring its melting temperature (Tₘ).

### BACKGROUND

The identification of nucleic acids in samples is important to many life science-related industries including basic research, clinical medicine, production of pharmaceuticals, food service, water supply, and environmental studies. In many instances, a target nucleic acid is difficult to amplify from a sample for any of a wide variety of reasons. These may include the nature of the sample (e.g., clean, dirty) or the target nucleic acid (e.g., low copy number, secondary structure, primer-dimers, non-specific amplification). It is typically useful to use a nucleic acid amplification reaction as a first step in analyzing a sample. However, in some instances, the cycle threshold (Cₜ) required to bring the amount of amplified nucleic acid to detectable (and reliable) levels is too high and leads to inconclusive or unreliable data. Accordingly, it is often very difficult to determine whether a sample actually does or does not contain the target nucleic acid. It is very important to have methods available that allow the skilled artisan to conclusively determine whether or not the target nucleic acid is present in a sample. Unlike currently available techniques, the methods described herein combine detection of an amplified product (i.e., quantitation of amplified nucleic acid) using a detectably labeled probe with confirmation of its identity via melting temperature (Tₘ) analysis. By combining these two powerful techniques, the user is able to conclusively determine whether a target nucleic acid is present within a sample.

### BRIEF DESCRIPTION OF THE DRAWINGS

FIG. 1: Exemplary TaqMelt™ assay system. A. TaqMelt™ assay system contains both an intercalating dye and a sequence-specific TaqMan® probe. B. Real-time fluorescence reading at the extension step generated an amplification plot containing signals from both the intercalating dye and the TaqMan® probe. C. Real-time fluorescence reading at or after denaturation generated the amplification signal from the TaqMan® probe only.

FIG. 2: Exemplary TaqMelt™ assays. Panels A and B show the Cₜ analysis using a HEXT™-labeled TaqMan® probe and the Tₘ analysis for a Staphylococcus aureus assay (Table 2). Panels C and D show the Cₜ analysis using a HEX™-labeled TaqMan® probe and the Tₘ analysis for a Salmonella enterica assay (Table 2).

FIG. 3: An example of a “borderline” TaqMan® assay result followed by dissociation curve (i.e., Tₘ) analysis. A. TaqMan® assay Cₜ values (VIC™ channel) are 35.5 (replicate 1) and 36.3 (replicate 2) suggesting borderline results of the assay with Cₜ threshold of 36. B. Melting curve analysis reveals the presence of the amplicon with an expected Tₘ of ˜79.6° C. (S. aureus) in both replicates. Thus, the TaqMelt™ assay results confirmed that the sample is “positive” for the assayed target.

FIG. 4: An example of an “inconclusive” TaqMan® assay followed by dissociation curve analysis. A. TaqMan® assay Cₜ values (VIC™ channel) are 38.0 (replicate 1) and Cₜ>40 (replicate 2) suggesting inconclusive results of the assay. B. Confirmatory melting curve analysis reveals the presence of the amplicon with an expected Tₘ of ˜83.9° C. (Salmonella enterica) in replicate 1. Thus, the TaqMelt™ assay results confirmed that the sample is actually “positive” for the Salmonella enterica.

FIG. 5: An example of multiplexing using TaqMelt™ that comprises a TaqMan® assay followed by dissociation curve analysis. Panels A and B show the Cₜ analysis using detectable label 1 (FAM™) and the Tₘ analysis for “Target 4”. Panels C and D show the Cₜ analysis using detectable label 2 (VIC™) and the Tₘ analysis for “Target 8” as in Table 1.

FIG. 6: Second exemplary multiplex assay (TaqMelt™) comprising a TaqMan® assay followed by dissociation curve analysis. A. The amplification curve was generated for the mixture of S. aureus (˜1000 cfu-s) and C. albicans (˜10 cfu-s) in duplex PCR format. Sequence-specific TaqMan® probes for both targets were labeled with HEX™ dye. B. Melting curve analysis reveals the presence of two melting peaks with Tₘ-s corresponding to both S. aureus (79.6° C.) and C. albicans (85.9° C.) amplicons. Thus, the TaqMelt™ assay results confirmed that the sample is “positive” for both targets.

FIG. 7: Third exemplary multiplex assay (TaqMelt™) comprising a TaqMan® assay followed by dissociation curve analysis. A. This exemplary assay displays the results using a sample containing S. aureus (˜100 cfu-s), Salmonella enterica (˜10,000 cfu-s) and C. albicans (˜100 cfu-s). B. Melting curve analysis reveals the presence of three melting peaks with Tₘ-s corresponding to S. aureus (79.6° C.), Salmonella enterica (83.9° C.) and C. albicans (85.9° C.) amplicons. Thus, the TaqMelt™ assay results confirmed that the sample is “positive” for all three targets.

### SUMMARY

Described herein are methods for detecting at least one target polynucleotide in a sample that comprise detecting an amplified nucleic acid sequence corresponding to the target polynucleotide using a detectable probe and analyzing the melting temperature of the amplified nucleic acid. These methods are herein denoted as TaqMelt™ assays. The methods comprise amplifying a nucleic acid corresponding to a target polynucleotide using at least one primer capable of hybridizing to said target polynucleotide and at least one oligonucleotide probe capable of hybridizing to said target polynucleotide 3′ relative to said primer. The probe typically comprises a detectable label (directly or indirectly bound to the probe) capable of being liberated during amplification. The amplification reaction typically occurs in the presence of a nucleic acid binding agent (i.e., an intercalating or non-intercalating agent) that binds to the double-stranded amplification product. The amplification reaction is typically monitored by detecting the signal emitted by the detectable label of the oligonucleotide probe. It is necessary for the nucleic acid binding agent to produce a detectable signal when bound to a double-stranded nucleic acid that is distinguishable from the signal produced when that same agent is in solution or bound to a single-stranded nucleic acid. The melting temperature (Tₘ) of the amplified nucleic acid is also determined by monitoring the release of the nucleic acid binding agent therefrom. Other embodiments of these methods are described herein.

The methods described herein are useful for detecting a variety of nucleic acids. Such nucleic acids include, for example, those of infectious agents, such as viruses, bacteria, parasites and pathogens, a disease process such as cancer or diabetes, or to measure an immune response. The methods described herein can also be organized as panels to detect multiple nucleic acids, such as where one or more infectious organisms are present in a sample (e.g., an environmental or food sample). In such cases, it may be beneficial to simultaneously query a sample for the presence of a group of infectious organisms by detecting target nucleic acids corresponding to one or more members of that group.

### DETAILED DESCRIPTION

Disclosed herein are methods and reagents for detecting at least one amplified target nucleic acid in a sample. The methods and reagents described herein can be used to identify target nucleic acids within a sample. The methods are typically utilized to identify an expressed nucleic acid, cell, tissue, or organism within a sample. In the process described herein, a sample is provided which contains, or is suspected of containing, a particular oligonucleotide sequence of interest, the “target nucleic acid.” The target may be RNA, DNA or an RNA/DNA hybrid. The target may be single-stranded or double-stranded. Target preparation is carried out in a manner appropriate for the particular amplification process to be implemented. For example, in a PCR method where the target nucleic acid is single-stranded RNA, such as mRNA, the target can be first reverse-transcribed into cDNA, prior to amplification. The methods described herein are useful for detecting a variety of nucleic acids. Such nucleic acids include, for example, those of infectious agents, such as viruses, bacteria, parasites and pathogens, a disease process such as cancer or diabetes, or to measure an immune response. Exemplary samples include biological samples such as a bodily fluid (e.g., blood, saliva, spinal fluid), a tissue sample, a food (e.g., meat) or beverage (e.g., milk) product and environmental samples (e.g., water). Expressed nucleic acids can include, for example, genes for which expression, or lack thereof, is associated with medical conditions such as infectious disease (i.e., bacterial, viral, fungal, protozoal infections) or cancer. The methods described herein can also be used to detect contaminants (i.e., bacteria, virus, fungus, or protozoan) in food or beverage products or environmental samples. Other uses for the methods described herein are also contemplated as will be understood by the skilled artisan.

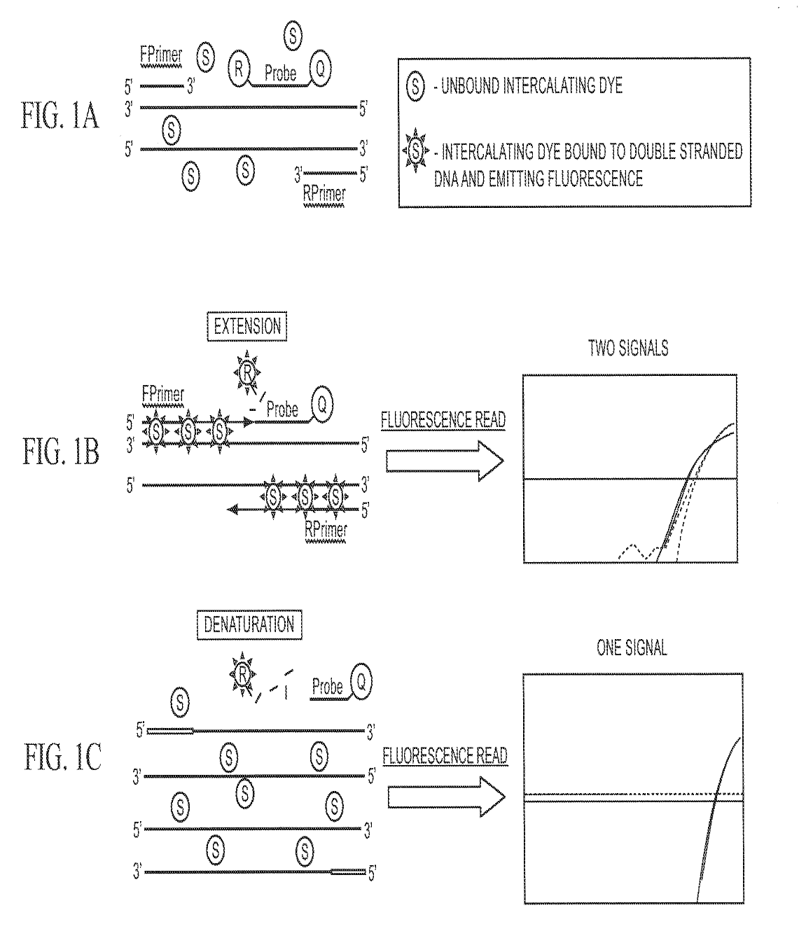

The methods described herein, denoted as TaqMelt™ assays, typically involve at least three steps: 1) amplifying a nucleic acid corresponding to a target nucleic acid; 2) detecting the amplified nucleic acid of step 1 using at least one probe having a detectable label; and, 3) confirming the presence of the amplified nucleic acid of step 1) by Tₘ analysis (FIG. 1). Step 1 is typically conducted prior to either of steps 2 and 3. Steps 2 and 3 can be completed simultaneously or in series. Thus, following amplification, the nucleic acids amplified thereby can be detected as in step 2 and then subjected to Tₘ analysis as in step 3; the nucleic acids amplified in the first step can be subjected to Tₘ analysis as in step 3 and then detected as in step 2 above; or, steps 2 and 3 can be performed simultaneously. The Tₘ analysis is typically dependent upon a nucleic acid binding agent that produces a detectable signal upon binding to double-stranded nucleic acid that is distinguishable from the signal produced when that same agent is in solution or bound to a single-stranded nucleic acid. Detection step 2 (i.e., detection of the probe) is typically performed under conditions in which detection of the nucleic acid binding agent is not favored (i.e., at a temperature at which the signals of the detectable label on the probe and the nucleic acid binding agent do not interfere with one another). Thus, the methods described herein utilize at least two steps to identify a target nucleic acid amplified from a sample. One step typically queries the sample by detecting the one or more detectable labels on the probe or probes. The other step typically confirms the presence or absence of the target nucleic acid in the sample by measuring the Tₘ of the amplified nucleic acids.

The combination of these reactions is particularly useful where the presence or absence of a target is questionable after the initial detection step. In many instances where the target nucleic acid is difficult to amplify for reasons related to the nature of the sample (e.g., clean, dirty) or the target nucleic acid (e.g., low copy number, secondary structure, primer-dimers, non-specific amplification), the cycle threshold (Cₜ) required to bring the amount of amplified nucleic acid to detectable (and reliable) levels is too high (i.e., >36). For similar reasons, it is also possible that a Tₘ analysis is inconclusive where, for instance, a particular amplified target nucleic acid may present multiple non-specific melting peaks. Accordingly, it is often very difficult to determine whether a sample actually does or does not contain the target nucleic acid. It is very important to have methods available that allow the skilled artisan to conclusively determine whether or not a sample contains the target nucleic acid. By combining detection of amplified product (i.e., quantitation of amplified nucleic acid) with confirmation of its identity via Tₘ analysis, one is able to make such conclusions.

The amplification reaction is typically performed using a nucleic acid polymerase, such as DNA polymerase, RNA polymerase, and reverse transcriptase, at least one oligonucleotide primer capable of specifically hybridizing to a target polynucleotide (from which the amplified target nucleic acid is amplified), at least one detectable probe that hybridizes to the amplified target nucleic acid, and which can be incorporated into the at least one primer), and at least one detectable nucleic acid binding agent (e.g., an intercalating or non-intercalating dye) which can be introduced before, during or after amplification. The probe typically contains a detectable label emitting a signal that can be monitored to ascertain whether the target nucleic acid has been amplified. In some embodiments, the probe is an oligonucleotide that hybridizes to the target nucleic acid 3′ relative to the at least one primer. In some embodiments, the polymerase has nuclease activity (i.e., 5′-to-3′ nuclease activity) for releasing the probe from the amplified nucleic acid. In some embodiments, release from the amplified nucleic acid renders the probe detectable. In some embodiments, the probe comprises a detectable label and a quencher molecule that quenches the detectable label when free but does not quench when the probe is hybridized to the amplified nucleic acid. In some embodiments, two or more probes can be used where at least one probe has a detectable label and at least one other probe has a quencher molecule. When in sufficiently close proximity to one another, the quencher molecule typically suppresses the signal of the detectable label on the other probe. In some embodiments, two or more probes, each having a different detectable label, can be used without quencher molecules. In such embodiments, the probes are rendered detectable, either de novo or by exhibiting a different signal than either probe alone, when in sufficiently close proximity to one another.

The Tₘ is typically measured by detecting the signal emitted by the detectable nucleic acid binding agent. The Tₘ of the amplified nucleic acid is typically known and is determined by particular characteristics of the amplified nucleic acid (e.g., length, G+C content). In some embodiments, the detectable nucleic acid binding agent is detectable only when bound to the double-stranded amplified target nucleic acid. Thus, when the amplified target nucleic acid reaches its Tₘ, the strands separate and the detectable nucleic acid binding agent is released therefrom. The resulting signal from the detectable nucleic acid binding agent is then accordingly decreased. As such, the presence of the amplified target nucleic acid is confirmed by detection of decreased fluorescence at the expected Tₘ of the amplified nucleic acid (e.g., the amplicon in a PCR reaction).

Any of several methods can be used to amplify the target nucleic acid from the sample. The term “amplifying” which typically refers to an “exponential” increase in the number of copies of the target nucleic acid is used herein to describe both linear and exponential increases in the numbers of a select target sequence of nucleic acid. The term “amplification reaction mixture” refers to an aqueous solution comprising the various reagents used to amplify a target nucleic acid. These include enzymes, including, but not limited to polymerases and thermostable polymerases such as DNA polymerase, RNA polymerase and reverse transcriptase, aqueous buffers, salts, amplification primers, target nucleic acid, and nucleoside triphosphates. Depending upon the context, the mixture can be either a complete or incomplete amplification reaction mixture. The method used to amplify the target nucleic acid can be any method available to one of skill in the art. Any in vitro means for multiplying the copies of a target sequence of nucleic acid can be utilized. These include linear, logarithmic, or any other amplification method. Exemplary methods include polymerase chain reaction (PCR; see, e.g., U.S. Pat. Nos. 4,683,202; 4,683,195; 4,965,188; and 5,035,996), isothermal procedures (using one or more RNA polymerases (see, e.g., WO 2006/081222), strand displacement (see, e.g., U.S. Pat. No. RE 39,007), partial destruction of primer molecules (see, e.g., WO 2006/087574)), ligase chain reaction (LCR) (see, e.g., Wu, et al. Genomics 4:560-569 (1990) and Barany, et al. Proc. Natl. Acad. Sci. USA 88:189-193 (1991)), Qβ RNA replicase systems (see, e.g., WO 1994/016108), RNA transcription-based systems (e.g., TAS, 3SR), rolling circle amplification (RCA) (see, e.g., U.S. Pat. No. 5,854,033; U.S. Pub. No. 2004/265897; Lizardi, et al. Nat. Genet. 19:225-232 (1998); and Banér, et al. Nucleic Acid Res. 26: 5073-5078 (1998)), and strand displacement amplification (SDA) (Little, et al. Clin. Chem. 45:777-784 (1999)), among others. Many systems are suitable for use in amplifying target nucleic acids and are contemplated herein as would be understood by one of skill in the art.

Any of several methods can be used to detect amplified target nucleic acids using primers or probes. Many different reagents, systems, or detectable labels can be used in the methods described herein. These include, for example, TaqMan® systems, detectable label-quencher systems (e.g., FRET, salicylate/DTPA ligand systems (see, e.g., Oser, et al. Angew. Chem. Int. Ed. Engl. 29:1167-1169 (1990), displacement hybridization, homologous probes, assays described in EP 070685), molecular beacons (e.g., NASBA), Scorpion, locked nucleic acid (LNA) bases (Singh, et al. Chem. Commun. 4:455-456 (1998)), peptide nucleic acid (PNA) probes (Pellestor, et al. Eur. J. Hum. Gen. 12:694-700 (2004)), Eclipse probes (Afonina, et al. Biotechniques 32:940-949 (2002)), light-up probes (Svanvik, et al. Anal. Biochem. 281:26-35 (2000)), molecular beacons (Tyagi, et al. Nat. Biotechnol. 14:303-308 (1996)), tripartite molecular beacons (Nutiu, et al. Nucleic Acids Res. 30:E94 (2002)), QuantiProbes (www.qiagen.com), HyBeacons (French, et al. Mol. Cell. Probes 15:363-374 (2001)), displacement probes (Li, et al. Nucleic Acids Res. 30:E5 (2002)), HybProbes (Cardullo, et al. Proc. Natl. Acad. Sci. USA 85:8790-8794 (1988)), MGB Alert (www.nanogen.com), Q-PNA (Fiandaca, et al. Genome Res. 11:609-613 (2001)), Plexor (www.Promega.com), LUX primers (Nazarenko, et al. Nucleic Acids Res. 30:E37 (2002)), Scorpion primers (Whitcombe, et al. Nat. Biotechnol. 17:804-807 (1999)), AmpliFluor® (Sunrise) primers (Nazarenko, et al. Nucleic Acids Res. 25:2516-2521 (1997)), DzyNA primers (Todd, et al. Clin. Chem. 46:625-630 (2000)), and the like. In each of these assays, the generation of amplification products can be monitored while the reaction is in progress. An apparatus for detecting the signal generated by the detectable label can be used to detect, measure, and quantify the signal before, during, or after amplification. The particular type of signal may dictate the choice of detection method. For example, in some embodiments, fluorescent dyes are used to label probes or amplified products. The probes bind to single-stranded or double-stranded amplified products, or the dyes intercalate into the double-stranded amplified products, and consequently, the resulting fluorescence increases as the amount of amplified product increases. In some embodiments, the Tₘ is ascertained by observing a fluorescence decrease as the double-stranded amplified product dissociates and the intercalating dye is released therefrom. The amount of fluorescence can be quantitated using standard equipment such as a spectra-fluorometer, for example. The use of other methods or reagents is also contemplated herein as would be understood by one of skill in the art.

One exemplary method for amplifying and detecting target nucleic acids is commercially available as TaqMan® (see, e.g., U.S. Pat. Nos. 4,889,818; 5,079,352; 5,210,015; 5,436,134; 5,487,972; 5,658,751; 5,538,848; 5,618,711; 5,677,152; 5,723,591; 5,773,258; 5,789,224; 5,801,155; 5,804,375; 5,876,930; 5,994,056; 6,030,787; 6,084,102; 6,127,155; 6,171,785; 6,214,979; 6,258,569; 6,814,934; 6,821,727; 7,141,377; and 7,445,900). TaqMan® assays are typically carried out by performing nucleic acid amplification on a target polynucleotide using a nucleic acid polymerase having 5′-to-3′ nuclease activity, a primer capable of hybridizing to said target polynucleotide, and an oligonucleotide probe capable of hybridizing to said target polynucleotide 3′ relative to said primer. The oligonucleotide probe typically includes a detectable label (e.g., a fluorescent reporter molecule) and a quencher molecule capable of quenching the fluorescence of said reporter molecule. Typically, the detectable label and quencher molecule are part of a single probe. As amplification proceeds, the polymerase digests the probe to separate the detectable label from the quencher molecule. The detectable label (e.g., fluorescence) is monitored during the reaction, where detection of the label corresponds to the occurrence of nucleic acid amplification (i.e., the higher the signal the greater the amount of amplification). Variations of TaqMan® assays, such as LNA™ spiked TaqMan® assay, are known in the art and would be suitable for use in the methods described herein.

Another exemplary system utilizes double-stranded probes in displacement hybridization methods (see, e.g., Morrison, et al. Anal. Biochem. 183:231-244 (1989); and Li, et al. (supra)). In such methods, the probe typically includes two complementary oligonucleotides of different lengths where one includes a detectable label and the other includes a quencher molecule. When not bound to a target nucleic acid, the quencher suppresses the signal from the detectable label. The probe becomes detectable upon displacement hybridization with a target nucleic acid. Multiple probes can be used, each containing different detectable labels, such that multiple target nucleic acids can be queried in a single reaction.

Additional exemplary methods for amplifying and detecting target nucleic acids involve “molecular beacons”, which are single-stranded hairpin shaped oligonucleotide probes. In the presence of the target sequence, the probe unfolds, binds and emits a signal (e.g., fluoresces). A molecular beacon typically includes at least four components: 1) the “loop”, an 18-30 nucleotide region which is complementary to the target sequence; 2) two 5-7 nucleotide “stems” found on either end of the loop and being complementary to one another; 3) at the 5′ end, a detectable label; and 4) at the 3′ end, a quencher dye that prevents the detectable label from emitting a single when the probe is in the closed loop shape (i.e., not bound to a target nucleic acid). Thus, in the presence of a complementary target, the “stem” portion of the beacon separates out resulting in the probe hybridizing to the target. Other types of molecular beacons are also known and can be suitable for use in the methods described herein. Molecular beacons can be used in a variety of assay systems. One such system is nucleic acid sequence-based amplification (NASBA®), a single step isothermal process for amplifying RNA to double stranded DNA without temperature cycling. A NASBA® reaction typically requires avian myeloblastosis virus (AMV), reverse transcriptase (RT), T7 RNA polymerase, RNase H, and two oligonucleotide primers. After amplification, the amplified target nucleic acid can be detected using a molecular beacon. Other uses for molecular beacons are known in the art and would be suitable for use in the methods described herein.

The Scorpion system is another exemplary assay format that can be used in the methods described herein. Scorpion primers are bi-functional molecules in which a primer is covalently linked to the probe, along with a detectable label (e.g., a fluorophore) and a quencher. In the presence of a target nucleic acid, the detectable label and the quencher separate which leads to an increase in signal emitted from the detectable label. Typically, a primer used in the amplification reaction includes a probe element at the 5′ end along with a “PCR blocker” element (such as an HEG monomer) at the start of the hairpin loop. The probe typically includes a self-complementary stem sequence with a detectable label at one end and a quencher at the other. In the initial amplification cycles, the primer hybridizes to the target and extension occurs due to the action of polymerase. The Scorpion system can be used to examine and identify point mutations using multiple probes with different tags to distinguish between the probes. Using PCR as an example, after one extension cycle is complete, the newly synthesized target region is attached to the same strand as the probe. Following the second cycle of denaturation and annealing, the probe and the target hybridize. The hairpin sequence then hybridizes to a part of the newly produced PCR product. This results in the separation of the detectable label from the quencher and causes emission of the signal. Other uses for molecular beacons are known in the art and would be suitable for use in the methods described herein.

One or more detectable labels or quenching agents are typically attached to a primer or probe. The detectable label can emit a signal when free or when bound to one the target nucleic acid. The detectable label can also emit a signal when in proximity to another detectable label. Detectable labels can also be used with quencher molecules such that the signal is only detectable when not in sufficiently close proximity to the quencher molecule. For instance, in some embodiments, the assay system can cause the detectable label to be liberated from the quenching molecule. Any of several detectable labels can be used to label the primers and probes used in the methods described herein. As mentioned above, in some embodiments the detectable label can be attached to a probe which can be incorporated into a primer or may otherwise bind to amplified target nucleic acid (for example, a detectable nucleic acid binding agent such as an intercalating or non-intercalating dye). When using more than one detectable label, each label should differ in its spectral properties such that the labels can be distinguished from each other, or such that together the detectable labels emit a signal that is not emitted by either detectable label alone. Exemplary detectable labels include, but are not limited to, a fluorescent dye or fluorophore (i.e., a chemical group that can be excited by light to emit fluorescence or phosphorescence), “acceptor dyes” capable of quenching a fluorescent signal from a fluorescent donor dye, and the like. Suitable detectable labels include, for example, fluorosceins (e.g., 5-carboxy-2,7-dichlorofluorescein; 5-Carboxyfluorescein (5-FAM); 5-HAT (Hydroxy Tryptamine); 6-HAT; 6-JOE; 6-carboxyfluorescein (6-FAM); FITC); Alexa fluors (e.g., 350, 405, 430, 488, 500, 514, 532, 546, 555, 568, 594, 610, 633, 635, 647, 660, 680, 700, 750); BODIPY fluorophores (e.g., 492/515, 493/503, 500/510, 505/515, 530/550, 542/563, 558/568, 564/570, 576/589, 581/591, 630/650-X, 650/665-X, 665/676, FL, FL ATP, FI-Ceramide, R6G SE, TMR, TMR-X conjugate, TMR-X, SE, TR, TR ATP, TR-X SE), coumarins (e.g., 7-amino-4-methylcoumarin, AMC, AMCA, AMCA-S, AMCA-X, ABQ, CPM methylcoumarin, coumarin phalloidin, hydroxycoumarin, CMFDA, methoxycoumarin), calcein, calcein AM, calcein blue, calcium dyes (e.g., calcium crimson, calcium green, calcium orange, calcofluor white), Cascade Blue, Cascade Yellow; Cy™ dyes (e.g., 3, 3.18, 3.5, 5, 5.18, 5.5, 7), cyan GFP, cyclic AMP Fluorosensor (FiCRhR), fluorescent proteins (e.g., green fluorescent protein (e.g., GFP. EGFP), blue fluorescent protein (e.g., BFP, EBFP, EBFP2, Azurite, mKalama1), cyan fluorescent protein (e.g., ECFP, Cerulean, CyPet), yellow fluorescent protein (e.g., YFP, Citrine, Venus, YPet), FRET donor/acceptor pairs (e.g., fluorescein/tetramethylrhodamine, IAEDANS/fluorescein, EDANS/dabcyl, fluorescein/fluorescein, BODIPY FL/BODIPY FL, Fluorescein/QSY7 and QSY9), LysoTracker and LysoSensor (e.g., LysoTracker Blue DND-22, LysoTracker Blue-White DPX, LysoTracker Yellow HCK-123, LysoTracker Green DND-26, LysoTracker Red DND-99, LysoSensor Blue DND-167, LysoSensor Green DND-189, LysoSensor Green DND-153, LysoSensor Yellow/Blue DND-160, LysoSensor Yellow/Blue 10,000 MW dextran), Oregon Green (e.g., 488, 488-X, 500, 514); rhodamines (e.g., 110, 123, B, B 200, BB, BG, B extra, 5-carboxytetramethylrhodamine (5-TAMRA), 5 GLD, 6-Carboxyrhodamine 6G, Lissamine, Lissamine Rhodamine B, Phallicidine, Phalloidine, Red, Rhod-2, 5-ROX (carboxy-X-rhodamine), Sulphorhodamine B can C, Sulphorhodamine G Extra, Tetramethylrhodamine (TRITC), WT), Texas Red, Texas Red-X, VIC and other labels described in, e.g., US Pub. No. 2009/0197254), among others as would be known to those of skill in the art. Other detectable labels can also be used (see, e.g., US Pub. No. 2009/0197254), as would be known to those of skill in the art.

Any of these systems and detectable labels, as well as many others, can be used to detect amplified target nucleic acids. The methods described herein allow for the presence of target nucleic acids to be detected and confirmed by combining at least two separate assay systems. In some embodiments, one system detects at least one target nucleic acid using a primer or probe, and the other by determining the Tₘ of the amplified target nucleic acid. As described above, these assays can be performed simultaneously or in series, optionally in combination with other methods, and in any order desired by the user. Described below are exemplary methods for measuring Tₘ.

The Tₘ of the amplified target nucleic acid is typically ascertained using a detectable nucleic acid binding agent (i.e., an intercalating agent or a non-intercalating agent). As used herein, an intercalating agent is an agent or moiety capable of non-covalent insertion between stacked base pairs of a double-stranded nucleic acid molecule. A non-intercalating agent is one that does not insert into the double-stranded nucleic acid molecule. The nucleic acid binding agent can produce a detectable signal directly or indirectly. The signal can be detectable directly using, for example, fluorescence or absorbance, or indirectly using, for example, any moiety or ligand that is detectably affected by its proximity to double-stranded nucleic acid, is suitable, for example, a substituted label moiety or binding ligand attached to the nucleic acid binding agent. It is necessary for the nucleic acid binding agent to produce a detectable signal when bound to a double-stranded nucleic acid that is distinguishable from the signal produced when that same agent is in solution or bound to a single-stranded nucleic acid. For example, intercalating agents such as ethidium bromide fluoresce more intensely when intercalated into double-stranded DNA than when bound to single-stranded DNA, RNA, or in solution (see, e.g., U.S. Pat. Nos. 5,994,056; 6,171,785; and 6,814,934). Similarly, actinomycin D fluoresces red when bound to single-stranded nucleic acids, and green when bound to double-stranded nucleic acids. And in another example, the photoreactive psoralen 4-aminomethyle-4-5′,8-trimethylpsoralen (AMT) has been reported to exhibit decreased absorption at long wavelengths and fluorescence upon intercalation into double-stranded DNA (Johnston et al. Photochem. Photobiol., 33:785-791 (1981). For example, U.S. Pat. No. 4,257,774 describes the direct binding of fluorescent intercalators to DNA (e.g., ethidium salts, daunomycin, mepacrine and acridine orange, 4′,6-diamidino-α-phenylindole). Non-intercalating agents (e.g., minor groove binders such as Hoechst 33258, distamycin, netropsin) can also be suitable for use. For example, Hoechst 33258 (Searle, et al. Nuc. Acids Res. 18:3753-3762 (1990)) exhibits altered fluorescence with an increasing amount of target. Other examples are available in the art that may be suitable for use in the methods described herein.

The detectable nucleic acid binding agent is typically present in the amplification reaction mixture during the amplification process but does not significantly inhibit the process. As amplification proceeds, the agent can produce a detectable signal. The detectable nucleic acid binding agent can be added to the reaction mixture before, during or after amplification, as desired by the user. For example, the detectable nucleic acid binding agent can be included in an amplification buffer comprising appropriate reagents, such as salts and buffering agents such that it is not necessary to separately add the binding agent to the amplification reaction. In certain embodiments, the detectable nucleic acid binding agent is detectable only when bound to double-stranded nucleic acids, such as the double-stranded amplified target nucleic acid. Thus, when the amplified target nucleic acid reaches its Tₘ, the detectable nucleic acid binding agent is released therefrom and its signal emission (e.g., fluorescence) is decreased. As such, the presence of the amplified target nucleic acid can be ascertained by detecting decreased signal emission at the expected Tₘ of the amplified target nucleic acid. In some embodiments, the Tₘ is defined as the point at which half the original signal emission intensity is observed. For example, when a fluorescent dye is used, Tₘ is defined as the temperature at which the fluorescence of the original sample (i.e., the amplified product) is decreased by 50 percent. This typically indicates that about 50 percent of the double-stranded amplified target nucleic acids have separated into single strands, thereby causing the release, or decreased or altered signal emission at a particular wavelength, of the dye. The Tₘ is a function of the composition of the amplified target nucleic acids including, for example, length, proportion of nucleotides that are either guanine or cytosine (i.e., “G+C composition”), and nucleotide modifications (e.g., LNA, inosine, GC tags). This is in contrast to previously described methods in which the Tₘ is defined as the temperature at which about 50 percent of the probes come off the amplified target nucleic acids, such as in FRET analysis. In such cases, the Tₘ relates to the probe composition, such as length and specific sequence but not the length of the amplified target nucleic acid or primer composition. Furthermore, the methods described herein also identify amplified target nucleic acids using two separate detection systems, one using a probe having a detectable label attached thereto and the other relying upon an intercalating dye that is incorporated into the amplified target nucleic acid.

Many suitable detectable nucleic acid binding agents are available to one of skill in the art and can be used alone or in combination with other agents or components of an assay system. Exemplary DNA binding agents may include, for example, acridines (e.g., acridine orange, acriflavine), actinomycin D (Jain, et al. J. Mol. Biol. 68:1-10 (1972)), anthramycin, BOBO™-1, BOBO™-3, BO-PRO™-1, cbromomycin, DAPI (Kapuściński, et al. Nuc. Acids Res. 6:3519-3534 (1979)), daunomycin, distamycin (e.g., distamycin D), dyes described in U.S. Pat. No. 7,387,887, ellipticine, ethidium salts (e.g., ethidium bromide), fluorcoumanin, fluorescent intercalators as described in U.S. Pat. No. 4,257,774, GelStar® (Cambrex Bio Science Rockland Inc., Rockland, Me.), Hoechst 33258 (Searle, et al. (supra)), Hoechst 33342, homidium, JO-PRO™-1, LIZ dyes, LO-PRO™-1, mepacrine, mithramycin, NED dyes, netropsin, 4′,6-diamidino-α-phenylindole, proflavine, POPO™-1, POPO™-3, PO-PRO™-1, propidium iodide, ruthenium polypyridyls, S5, SYBR® Gold, SYBR® Green I (U.S. Pat. Nos. 5,436,134 and 5,658,751), SYBR® Green II, SYTOX blue, SYTOX green, SYTO® 43, SYTO® 44, SYTO® 45, SYTOX® Blue, TO-PRO®-1, SYTO® 11, SYTO® 13, SYTO® 15, SYTO® 16, SYTO® 20, SYTO® 23, thiazole orange (Aldrich Chemical Co., Milwaukee, Wis.), TOTO™-3, YO-PRO®-1, and YOYO®-3 (Molecular Probes, Inc., Eugene, Oreg.), among others. SYBR® Green I (see, e.g., U.S. Pat. Nos. 5,436,134; 5,658,751; and 6,569,927), for example, has been used to monitor a PCR reaction by amplifying the target sequence in the presence of the dye, exciting the biological sample with light at a wavelength absorbed by the dye and detecting the emission therefrom; and, determining a melting profile of the amplified target sequence. The presence of amplified products and, therefore, the target sequence in the sample, can thereafter be determined by, for example, performing a melting curve analysis (i.e., non-linear least squares regression of the sum of multiple gaussians). It is to be understood that the use of the SYBR® Green dye is presented as an example, and that many such dyes can be used in the methods described herein. Other nucleic acid binding agents can also be suitable as would be understood by one of skill in the art.

The methods described herein are useful for detecting a variety of nucleic acids. Such nucleic acids include, for example, those of infectious agents, such as viruses, bacteria, parasites and pathogens, a disease process such as cancer or diabetes, or to measure an immune response. The methods described herein can also be organized as panels to detect multiple nucleic acids, such as where one or more infectious organisms are present in a sample (e.g., an environmental or food sample). In such cases, it may be beneficial to simultaneously query a sample for the presence of a group of infectious organisms by detecting target nucleic acids corresponding to one or more members of that group.

The TaqMelt™ assays described herein can be used to analyze samples for the presence of infectious, pathogenic or parasitic agents in, for example, biological or environmental samples. For example, the methods disclosed herein can be used in food safety analysis to monitor levels of pathogens, such as E. coli O157:H7 in meat and produce items and Salmonella spp. in meat, produce and beverage items. Furthermore, the assays described herein can be used to monitor the quality of drinking water supplies and sources for pathogens and infectious or parasitic agents that can arise from various sources of contamination, such as by water runoff from farms, sewage or refuse facility leaks and deliberate contamination.

In addition, the methods disclosed herein can be used to analyze biological samples from humans and other animals for detection of infectious, pathogenic or parasitic agent. For example, blood or other biological samples can be tested or monitored for viruses, such as H1N1 and ebola virus, during suspected outbreaks to ascertain the extent of infection and spread. The TaqMelt™ assays described herein can also be used to test non-human animal biological samples, such as avian or pig samples, for infectious or pathogenic agents, such as avian and swine flu viruses, respectively. Information gathered from these assays can be used to detect the extent of infection during outbreaks of diseases in non-human animals.

Exemplary viruses that can be detected using the methods described herein include, but are not limited to, a dsDNA virus (e.g. adenovirus, herpesvirus, epstein-barr virus, herpes simplex type 1, herpes simplex type 2, human herpes virus simplex type 8, human cytomegalovirus, varicella-zoster virus, poxvirus); ssDNA virus (e.g., parvovirus, papillomavirus (e.g., E1, E2, E3, E4, E5, E6, E7, E8, BPV1, BPV2, BPV3, BPV4, BPV5 and BPV6 (In Papillomavirus and Human Cancer, edited by H. Pfister (CRC Press, Inc. 1990); Lancaster et al., Cancer Metast. Rev. 6:6653-6664 (1987); Pfister, Adv. Cancer Res. 48:113-147 (1987)); dsRNA viruses (e.g., reovirus); (+)ssRNA viruses (e.g., picornavirus, coxsackie virus, hepatitis A virus, poliovirus, togavirus, rubella virus, flavivirus, hepatitis C virus, yellow fever virus, dengue virus, west Nile virus); (−)ssRNA viruses (e.g., orthomyxovirus, influenza virus, rhabdovirus, paramyxovirus, measles virus, mumps virus, parainfluenza virus, respiratory syncytial virus, rhabdovirus, rabies virus); ssRNA-RT viruses (e.g. retrovirus, human immunodeficiency virus (HIV)); and, dsDNA-RT viruses (e.g. hepadnavirus, hepatitis B). Other viruses not listed above can also be detected as would be understood by one of skill in the art.

Nucleic acids of one or more bacterial species (spp.) may be detected such as, for example, Bacillus spp. (e.g., Bacillus anthracis), Bordetella spp. (e.g., Bordetella pertussis), Borrelia spp. (e.g., Borrelia burgdorferi), Brucella spp. (e.g., Brucella abortus, Brucella canis, Brucella melitensis, Brucella suis), Campylobacter spp. (e.g., Campylobacter jejuni), Chlamydia spp. (e.g., Chlamydia pneumoniae, Chlamydia psittaci, Chlamydia trachomatis), Clostridium spp. (e.g., Clostridium botulinum, Clostridium difficile, Clostridium perfringens, Clostridium tetani), Corynebacterium spp. (e.g., Corynebacterium diptheriae), Enterococcus spp. (e.g., Enterococcus faecalis, Enterococcus faecum), Escherichia spp. (e.g., Escherichia coli), Francisella spp. (e.g., Francisella tularensis), Haemophilus spp. (e.g., Haemophilus influenza), Helicobacter spp. (e.g., Helicobacter pylori), Legionella spp. (e.g., Legionella pneumophila), Leptospira spp. (e.g., Leptospira interrogans), Listeria spp. (e.g., Listeria monocytogenes), Mycobacterium spp. (e.g., Mycobacterium leprae, Mycobacterium tuberculosis), Mycoplasma spp. (e.g., Mycoplasma pneumoniae), Neisseria spp. (e.g., Neisseria gonorrhea, Neisseria meningitidis), Pseudomonas spp. (e.g., Pseudomonas aeruginosa), Rickettsia spp. (e.g., Rickettsia rickettsii), Salmonella spp. (e.g., Salmonella enterica, Salmonella typhi, Salmonella typhinurium), Shigella spp. (e.g., Shigella sonnei), Staphylococcus spp. (e.g., Staphylococcus aureus, Staphylococcus epidermidis, Staphylococcus saprophyticus, coagulase negative staphylococcus (e.g., U.S. Pat. No. 7,473,762)), Streptococcus spp. (e.g., Streptococcus agalactiae, Streptococcus pneumoniae, Streptococcus pyrogenes), Treponema spp. (e.g., Treponema pallidum), Vibrio spp. (e.g., Vibrio cholerae), and Yersinia spp. (e.g., Yersinia pestis). Other bacterial species not listed above can also be detected as would be understood by one of skill in the art.

Nucleic acids of one or more fungal species (spp.) can be detected such as, for example, Actinomyces spp. (e.g., A. israelii, A. bovis, A. naeslundii), Allescheria spp. (e.g., A. boydii), Aspergillus spp. (e.g., A. fumigatus, A. nidulans), Blastomyces spp. (e.g., B. dermatidis), Candida spp. (e.g., C. albicans), Cladosporium spp. (e.g., C. carrionii), Coccidioides spp. (e.g., C. immitis), Cryptococcus spp. (e.g., C. neoformans), Fonsecaea spp. (e.g., F. pedrosoi, F. compacta, F. dermatidis), Histoplasma spp. (e.g., H. capsulatum), Nocardia spp. (e.g., N. asteroids, N. brasiliensis), Keratinomyces spp. (e.g., K. ajelloi), Madurella spp. (e.g., M. grisea, M. mycetomi), Microsporum spp. (e.g., M. adnouini, M. gypseum, M. canis), Mucor spp. (e.g., M. corymbifer, Absidia corymbifera), Paracoccidioides spp. (e.g., P. brasiliensis), Phialosphora spp. (e.g., P. jeansilmei, P. verrucosa), Rhizopus spp. (e.g., R. oryzae, R. arrhizus, R. nigricans), Sporotrichum spp. (e.g., S. Schenkii), and Trichophyton spp. (e.g., T. mentagrophytes, T. rubrum). Other fungal species not listed above can also be detected as would be understood by one of skill in the art.

Nucleic acids of one or more one or more parasitic organisms (spp.) can also be detected such as, for example, Ancylostoma spp. (e.g., A. duodenale), Anisakis spp., Ascaris lumbricoides, Balantidium coli, Cestoda spp., Cimicidae spp., Clonorchis sinensis, Dicrocoelium dendriticum, Dicrocoelium hospes, Diphyllobothrium latum, Dracunculus spp., Echinococcus spp. (e.g., E. granulosus, E. multilocularis), Entamoeba histolytica, Enterobius vermicularis, Fasciola spp. (e.g., F. hepatica, F. magna, F. gigantica, F. jacksoni), Fasciolopsis buski, Giardia spp. (Giardia lamblia), Gnathostoma spp., Hymenolepis spp. (e.g., H. nana, H. diminuta), Leishmania spp., Loa boa, Metorchis spp. (M. conjunctus, M. albidus), Necator americanus, Oestroidea spp. (e.g., botfly), Onchocercidae spp., Opisthorchis spp. (e.g., O. viverrini, O. felineus, O. guayaquilensis, and O. noverca), Plasmodium spp. (e.g., P. falciparum), Protofasciola robusta, Parafasciolopsis fasciomorphae, Paragonimus westermani, Schistosoma spp. (e.g., S. mansoni, S. japonicum, S. mekongi, S. haematobium), Spirometra erinaceieuropaei, Strongyloides stercoralis, Taenia spp. (e.g., T. saginata, T. solium), Toxocara spp. (e.g., T. canis, T. cati), Toxoplasma spp. (e.g., T. gondii), Trichobilharzia regenti, Trichinella spiralis, Trichuris trichiura, Trombiculidae spp., Trypanosoma spp., Tunga penetrans, or Wuchereria bancrofti. Other species of parasite not listed above can also be detected as would be understood by one of skill in the art.

The assays described herein, can also be used to detect or diagnose diseases. For example, biological samples can be analyzed for cancer-specific markers, genetic polymorphisms and mutations indicative of certain types of cancer, in order to detect the presence of a cancer, determine the stage or progression of the disease, determine the prognosis of the disease or determine a course of treatment based on the genetic makeup of the tumor or disease. To detect a disease process such as cancer, nucleic acids encoding one or more tumor antigens can be detected, where a cancerous cell is the source of the antigen. Exemplary tumor antigens that could be detected include, for example, cancer-testis (CT) antigens (i.e., MAGE, NY-ESO-1); melanocyte differentiation antigens (i.e., Melan A/MART-1, tyrosinase, gp100); mutational antigens (i.e., MUM-1, p 53, CDK-4); overexpressed ‘self’ antigens (i.e., HER-2/neu, p 53); and, viral antigens (i.e., HPV, EBV). Suitable TAs include, for example, gp100 (Cox, et al. Science 264:716-719 (1994)), MART-1/Melan A (Kawakami, et al. J. Exp. Med. 180:347-352 (1994)), gp75 (TRP-1) (Wang, et al. J. Exp. Med. 183:1131-1140 (1996)), tyrosinase (Wölfel, et al. Eur. J. Immunol. 24:759-764 (1994)), NY-ESO-1 (WO 1998/014464; WO 1999/018206), melanoma proteoglycan (Hellström, et al. J. Immunol. 130:1467-1472 (1983)), MAGE family antigens (i.e., MAGE-1,2,3,4,6, and 12; Van der Bruggen, et al. Science 254:1643-1647 (1991); U.S. Pat. No. 6,235,525), BAGE family antigens (Boël, et al. Immunity 2:167-175 (1995)), GAGE family antigens (i.e., GAGE-1,2; Van den Eynde, et al. J. Exp. Med. 182:689-698 (1995); U.S. Pat. No. 6,013,765), RAGE family antigens (i.e., RAGE-1; Gaugler, et al. Immunogenetics 44:323-330 (1996); U.S. Pat. No. 5,939,526), N-acetylglucosaminyltransferase-V (Guilloux, et al. J. Exp. Med. 183:1173-1183 (1996)), p 15 (Robbins, et al. J. Immunol. 154:5944-5950 (1995)), β-catenin (Robbins, et al. J. Exp. Med. 183:1185-1192 (1996)), MUM-1 (Coulie, et al. Proc. Natl. Acad. Sci. USA, 92:7976-7980 (1995)), cyclin dependent kinase-4 (CDK4) (Wölfel, et al. Science 269:1281-1284 (1995)), p 21-ras (Fossum, et al. Int. J. Cancer 56:40-45 (1994)), BCR-abl (Bocchia, et al. Blood 85:2680-2684 (1995)), p 53 (Theobald, et al. Proc. Natl. Acad. Sci. USA, 92:11993-11997 (1995)), p 185 HER2/neu (erb-B1; Fisk, et al. J. Exp. Med. 181:2109-2117 (1995)), epidermal growth factor receptor (EGFR) (Harris, Breast Cancer Res. Treat. 29:1-2 (1994)), carcinoembryonic antigens (CEA) (Kwong, et al. J. Natl. Cancer Inst. 85:982-990 (1995) U.S. Pat. Nos. 5,756,103; 5,274,087; 5,571,710; 6,071,716; 5,698,530; 6,045,802; EP 263933; EP 346710; and, EP 784483); carcinoma-associated mutated mucins (i.e., MUC-1 gene products; Jerome, et al. J. Immunol. 151:1654-1662 (1993)); EBNA gene products of EBV (i.e., EBNA-1; Rickinson, et al. Cancer Surveys 13:53-80 (1992)); E7, E6 proteins of human papillomavirus (Ressing, et al. J. Immunol. 154:5934-5943 (1995)); prostate specific antigen (PSA; Xue, et al. Prostate, 30:73-78 (1997)); prostate specific membrane antigen (PSMA; Israeli, et al. Cancer Res. 54:1807-1811 (1994)); idiotypic epitopes or antigens, for example, immunoglobulin idiotypes or T cell receptor idiotypes (Chen, et al. J. Immunol. 153:4775-4787 (1994)); KSA (U.S. Pat. No. 5,348,887), kinesin 2 (Dietz, et al. Biochem. Biophys. Res. Commun. 275:731-738 (2000), HIP-55, TGFβ-1 anti-apoptotic factor (Toomey, et al. Br. J. Biomed. Sci. 58:177-183 (2001), tumor protein D52 (Bryne, et al. Genomics 35:523-532 (1996)), H1FT, NY-BR-1 (WO 2001/047959), NY-BR-62, NY-BR-75, NY-BR-85, NY-BR-87 and NY-BR-96 (Scanlan, M. Serologic and Bioinformatic Approaches to the Identification of Human Tumor Antigens, in Cancer Vaccines 2000, Cancer Research Institute, New York, N.Y.), or pancreatic cancer antigens (e.g., SEQ ID NOS: 1-288 of U.S. Pat. No. 7,473,531). Other tumor antigens not listed above can also be detected as would be understood by one of skill in the art.

The methods described herein can also be used to detect or measure an immune response associated with one or more of an effect (e.g., maturation, proliferation, direct- or cross-presentation of antigen, gene expression profile) on cells of either the innate or adaptive immune system. For example, the immune response may involve, effect, or be detected in innate immune cells such as, for example, dendritic cells, monocytes, macrophages, natural killer cells, or granulocytes (e.g., neutrophils, basophils or eosinophils). The immune response may also involve, effect, or be detected in adaptive immune cells including, for example, lymphocytes (e.g., T cells or B cells). The immune response can be observed by detecting such involvement or effects including, for example, the presence, absence, or altered (e.g., increased or decreased) expression or activity of one or more immunomodulators such as a hormone, cytokine, interleukin (e.g., any of IL-1 through IL-35), interferon (e.g., any of IFN-I (IFN-α, IFN-β, IFN-ε, IFN-κ, IFN-τ, IFN-ζ, IFN-ω), IFN-II (e.g., IFN-γ), IFN-III (IFN-λ1, IFN-λ2, IFN-λ3)), chemokine (e.g., any CC cytokine (e.g., any of CCL1 through CCL28), any CXC chemokine (e.g., any of CXCL1 through CXCL24), Mip 1 a), any C chemokine (e.g., XCL1, XCL2), any CX3C chemokine (e.g., CX3CL1)), tumor necrosis factor (e.g., TNF-α, TNF-β)), negative regulators (e.g., PD-1, IL-T) or any of the cellular components (e.g., kinases, lipases, nucleases, transcription-related factors (e.g., IRF-1, IRF-7, STAT-5, NFKB, STAT3, STAT1, IRF-10), or cell surface markers suppressed or induced by such immunomodulators) involved in the expression of such immunomodulators. The presence, absence or altered expression can be detected within cells of interest or near those cells (e.g., within a cell culture supernatant, nearby cell or tissue in vitro or in vivo, or in blood or plasma). Other immune regulators, effectors, or the like not listed above can also be detected as would be understood by one of skill in the art.

As mentioned above, the methods described herein can be used to simultaneously screen a sample for the presence of one or more target nucleic acids. An exemplary matrix providing for the use of multiple detectable labels and association of those labels with a particular Tₘ is outlined in Table 1.

TABLE 1

| | | | | | Target |
|---|---|---|---|---|---|
| | Detectable Label (e.g. fluorescent label) | | | | nucleic acid |
| Target* | A | B | C | D | Tₘ |
| T1 | √ | | | | 1 |
| T2 | √ | | | | 2 |
| T3 | √ | | | | 3 |
| T4 | √ | | | | 4 |
| T5 | | √ | | | 1 |
| T6 | | √ | | | 2 |
| T7 | | √ | | | 3 |
| T8 | | √ | | | 4 |
| T9 | | | √ | | 1 |
| T10 | | | √ | | 2 |
| T11 | | | √ | | 3 |
| T12 | | | √ | | 4 |
| T13 | | | | √ | 1 |
| T14 | | | | √ | 2 |
| T15 | | | | √ | 3 |
| T16 | | | | √ | 4 |

In Table 1, each of T1-T16 represents a different target nucleic acid that can be amplified from a sample; A, B, C and D represent different detectable labels on the probes for each target nucleic acid; and Tₘ 1, 2, 3 and 4 each represent a different Tₘ for each amplified target nucleic acid. It should be understood that more or less detectable labels or Tₘ can be selected, and that detectable labels A-D and Tₘ 1-4 are presented merely for explanatory purposes. Typically, detectable labels A, B, C and D are distinguishable from one another in that, for example, each emits a different signal (e.g., fluoresce at a different wavelength). The Tₘ of the amplified target nucleic acids can be the same or different where different detectable labels are used in a reaction. However, the Tₘ of the amplified target nucleic acids typically differ when the same detectable labels are used in a reaction. Thus, to simultaneously detect multiple target nucleic acids, either the detectable label on the probe or the Tₘ must differ to distinguish between amplified target nucleic acids. For example, T1, T2, T3, and T4 can all be queried using the same detectable label on their respective probes (e.g., detectable label A) but must each exhibit a different Tₘ to be distinguishable from one another. Thus, the methods disclosed herein also allow the analysis of one, two, or multiple (i.e., more than two) samples per reaction.

Exemplary primers and probes that can be used to assay a sample for the presence of S. aureus, S. enterica, and C. albicans are shown in Table 2.

TABLE 2

| Assay | Sequence 5′-3′ | Reference |
|---|---|---|
| S. aureus | | Journal of |
| Forward | CGCTACTAGTTGCTTAGTGTTAACTTTAGTTG | Applied |
| Primer | (SEQ ID NO.: 1) | Microbiology |
| Reverse | TGCACTATATACTGTTGGATCTTCAGAA | ISSN 1364-5072 |
| Primer | (SEQ ID NO.: 2) | |
| Probe | (HEX)TGCATCACAAACAGATAACGGCGTAAATAGAAG(NFQ) | |
| | (SEQ ID NO.: 3) | |
| S. enterica | | App. Env. |
| Forward | CTCACCAGGAGATTACAACATGG (SEQ ID NO.: 4) | Microb. p. 7046- |
| Primer | | 7052 (Dec. 2004) |
| Reverse | AGCTCAGACCAAAAGTGACCATC (SEQ ID NO.: 5) | |
| Primer | | |
| Probe | (HEX) CACCGACGGCGAGACCGACTTT (NFQ) | |
| | (SEQ ID NO.: 6) | |
| C. albicans | | Anal. Chem. |
| Forward | CTGTTTGAGCGTCGTTTC (SEQ ID NO.: 7) | 2010, 82, 2310- |
| Primer | | 2316 |
| Reverse | ATGCTTAAGTTCAGCGGGTAG (SEQ ID NO.: 8) | |
| Primer | | |
| Probe | (HEX)CTGGGTTTGGTGTTGAGCAATACG(NFQ) | |
| | (SEQ ID NO.: 9) | |

A sample can be assayed for the presence of any one or more of such organisms individually, or as part of a panel of more than one of such organisms.

An exemplary panel of organisms that may be selected to screen food-related products is shown in Table 3:

TABLE 3

| Organism | Tₘ | Probe dye |
|---|---|---|
| Listeria monocytogenes | 75.8 | VIC ™ |
| Staph. aureus | 75.5 | FAM ™ |
| C. jejuni | 75.9 | NED ™ |
| V. cholerae | 78 | FAM ™ |
| E. coli O157:H7 | 79.7 | VIC ™ |
| Salmonella spp. | 82.5 | VIC ™ |
| E. sakazakii | 84.5 | NED ™ |
| V. parahaemolyticus | 84.5 | FAM ™ |
| V. vulnificus | 84.7 | VIC ™ |

A sample can be assayed for the presence of any one or more of such organisms individually, or as part of a panel of more than one of such organisms, using probes having distinguishable Tₘ values, such as those shown in Table 3.

An exemplary panel that includes microorganisms to be screened for in pharmaceutical-related products is shown in Table 4:

TABLE 4

| Organism | Tₘ | Probe dye |
|---|---|---|
| Staph. aureus | 75.5 | VIC ™ |
| C. albicans | 75 | FAM ™ |
| E. coli | 75 | NED ™ |
| C. jejuni | 75.9 | LIZ ™ |
| A. niger | 77.9 | FAM ™ |
| V. cholerae | 78 | VIC ™ |
| Salmonella spp. | 82.5 | VIC ™ |
| E. sakazakii | 84.5 | FAM ™ |
| P. aeruginosa | 87 | NED ™ |

A sample can be assayed for the presence of any one or more of such organisms individually, or as part of a panel of more than one of such organisms using probes having distinguishable Tₘ values, such as those shown in Table 4.

Kits for performing the methods described herein are also provided. For use with a PCR-based assay, the kit typically includes at least a set of primers for amplifying at least one target nucleic acid from a sample, or the corresponding one or more probes labeled with a detectable label. The kit can also include samples containing pre-defined target nucleic acids to be used in control reactions. The kit can also optionally include stock solutions, buffers, enzymes, detectable labels or reagents required for detection, tubes, membranes, and the like that may be used to complete the amplification reaction. In some embodiments, multiple primer sets are included. Other embodiments of particular systems and kits are also contemplated which would be understood by one of skill in the art.

All references cited within this disclosure are hereby incorporated by reference in their entirety. Certain embodiments are further described in the following examples. These embodiments are provided as examples only and are not intended to limit the scope of the claims in any way.

### EXAMPLES

The assays described herein involve at least two steps. The first step involves amplifying a nucleic acid and the second step involves detecting the amplified nucleic acids. In these Examples, the amplification step is accomplished using a real-time PCR assay (e.g., TaqMan® assay) performed in the presence of a DNA binding agent (e.g., SYBR® Green, dissociation curve analysis). Such assays are referred to in these examples as TaqMelt™ assays. The real-time PCR assays were run on Applied Biosystems 7500 or 7500 Fast Real-Time PCR System with the run mode set to “Fast 7500”. The assays described in these Examples utilized one or both of the detectable labels FAM™ and VIC™. Primer and probe concentrations were adjusted as necessary for a particular assay. The Tₘ was determined using a standard algorithm and ramping speed of the AB 7500 instrument during the Dissociation Stage analysis. Dissociation curves provide information relating to Tₘ of the amplified targets at the X-axis and also the derivative values of fluorescence at the Y-axis. The primers and probes used to assay samples for the presence of S. aureus, C. albicans, and S. enterica are shown above in Table 2.

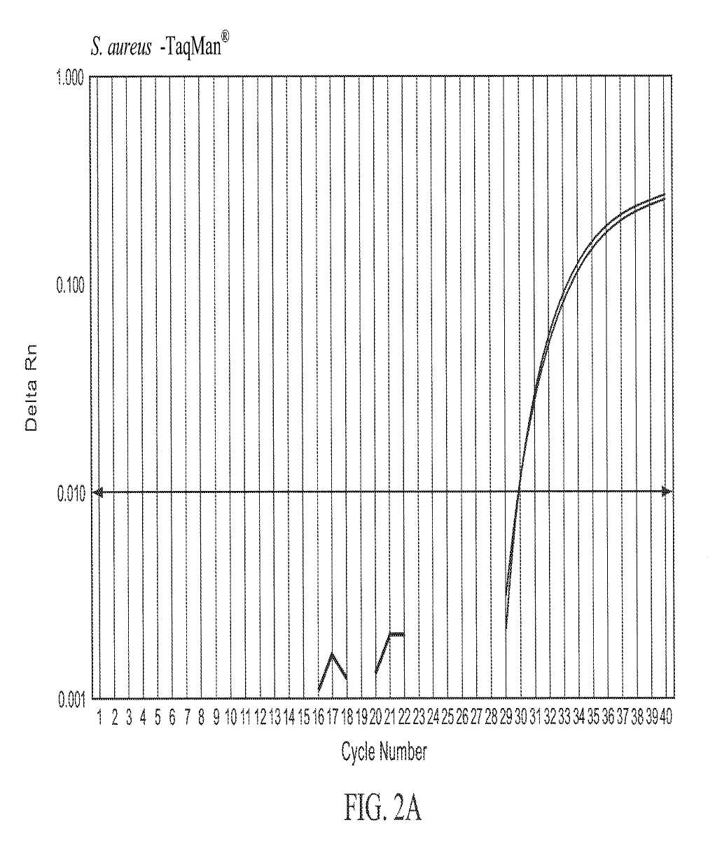

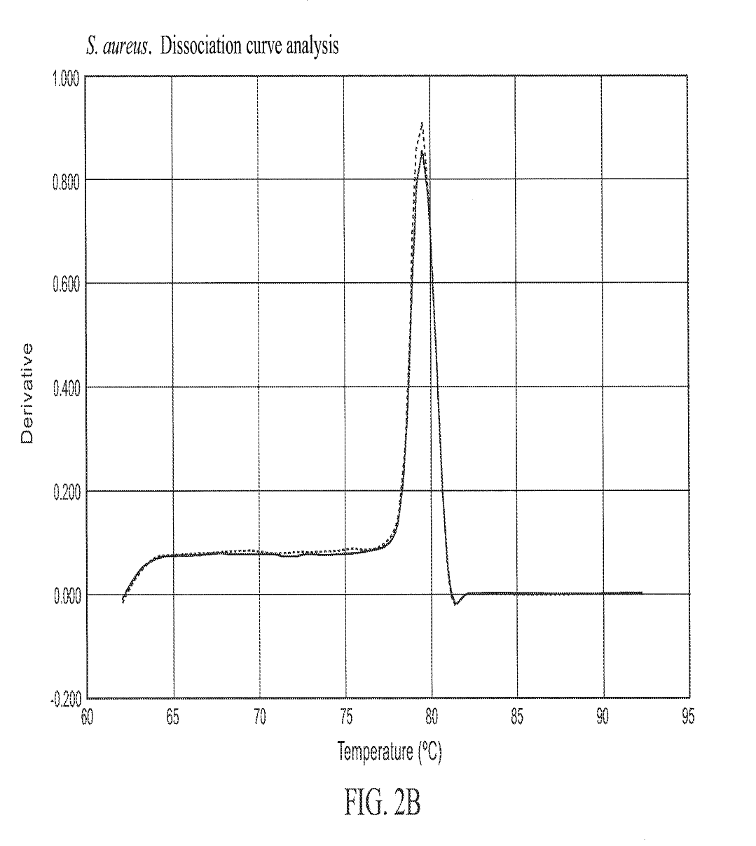

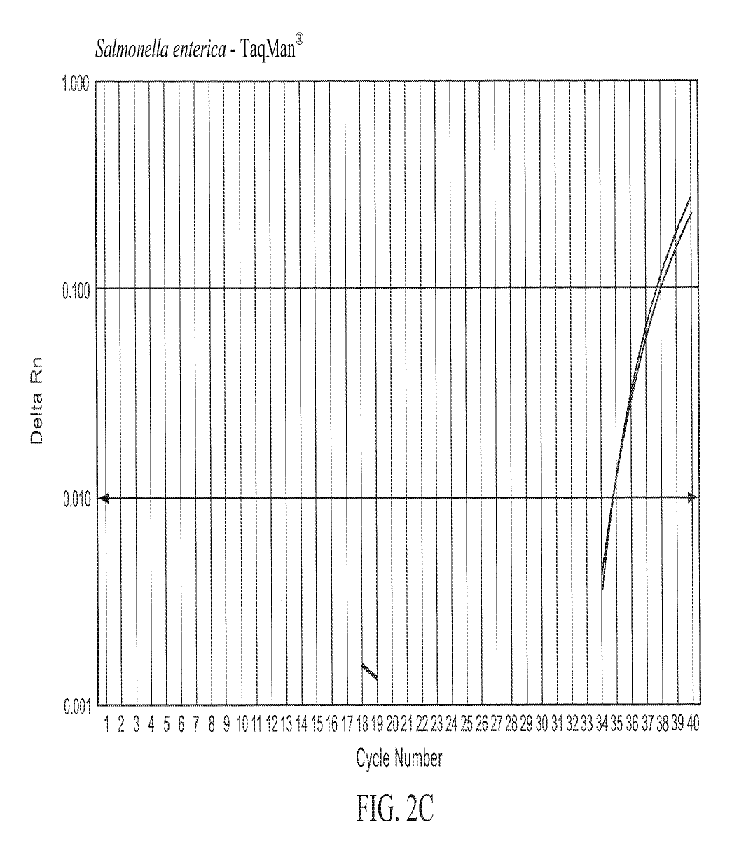

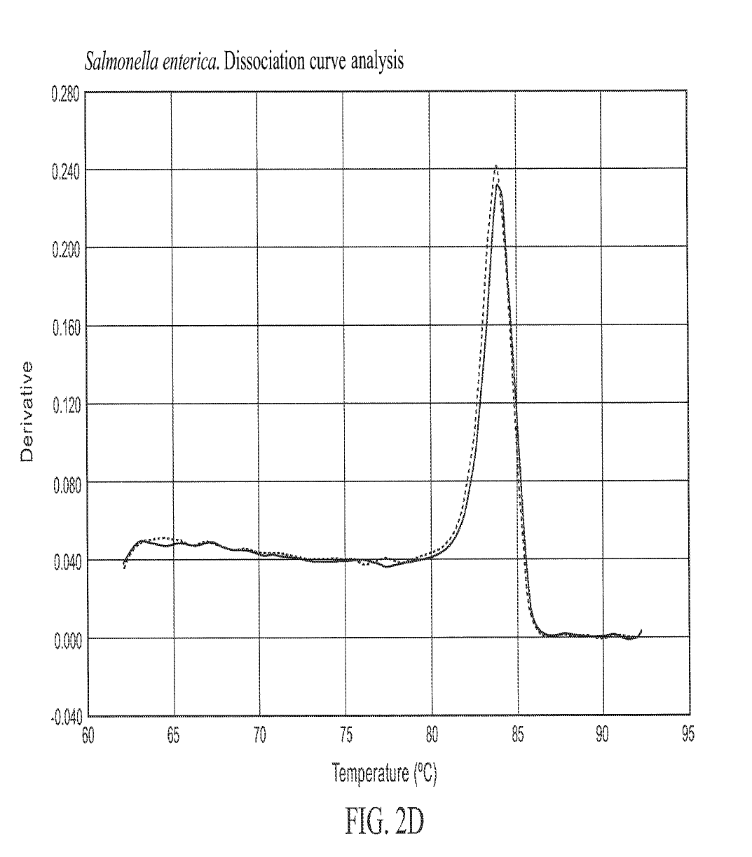

The results of an exemplary TaqMelt™ assay used to detect two different organisms (Staphylococcus aureus, Salmonella enterica) is illustrated in FIG. 2. Real-time PCR reactions were performed with 5 pg genomic S. aureus genomic DNA (˜1,000 cfu) and 0.5 pg of Salmonella enterica genomic DNA. PCR reactions were prepared in 1×X PCR reaction buffer (20 mM Tris, 50 mM KCl pH 8.4, 250 nM each dNTPs, 5 mM MgCl₂, 2.5 uM SYTO®-9, 90 nM ROX (passive reference) and 2.5 U of Platinum®Taq DNA polymerase. Real-time PCR cycling was performed on AB 7500 FAST real time PCR system as follows: 95° C. for 2 min; 40 cycles of 95° C. for 30 seconds (fluorescence reading on) and 60° C. for 15 seconds in Fast 7500 mode. The fluorescence data were collected in optical channels A and B (FAM™/SYBR® Green, VIC™) during the PCR thermocycling and in optical channel A (FAM™/SYBR® Green) during the melting curve analysis. Primer and probe concentrations used in all experiments were 300 nM and 150 nM respectively. Melting temperature of each amplicons was determined using a standard algorithm and ramping speed of the AB 7500 instrument during the Dissociation Stage analysis. Dissociation curves provide the information of the melting temperature (Tₘ) of the amplified targets at the X-axis and also the derivative values of fluorescence at the Y-axis.

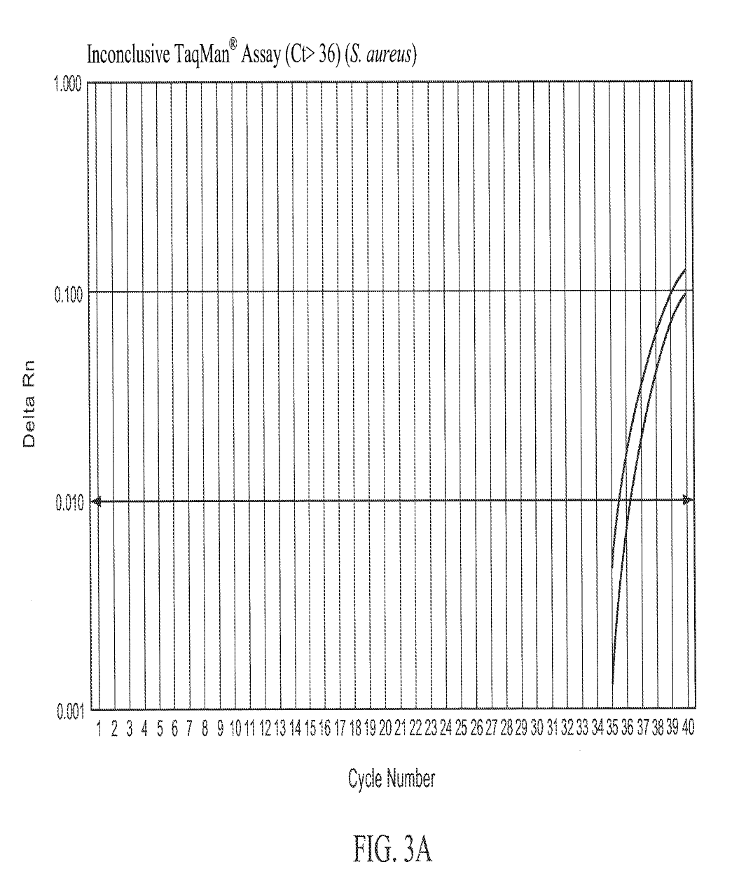

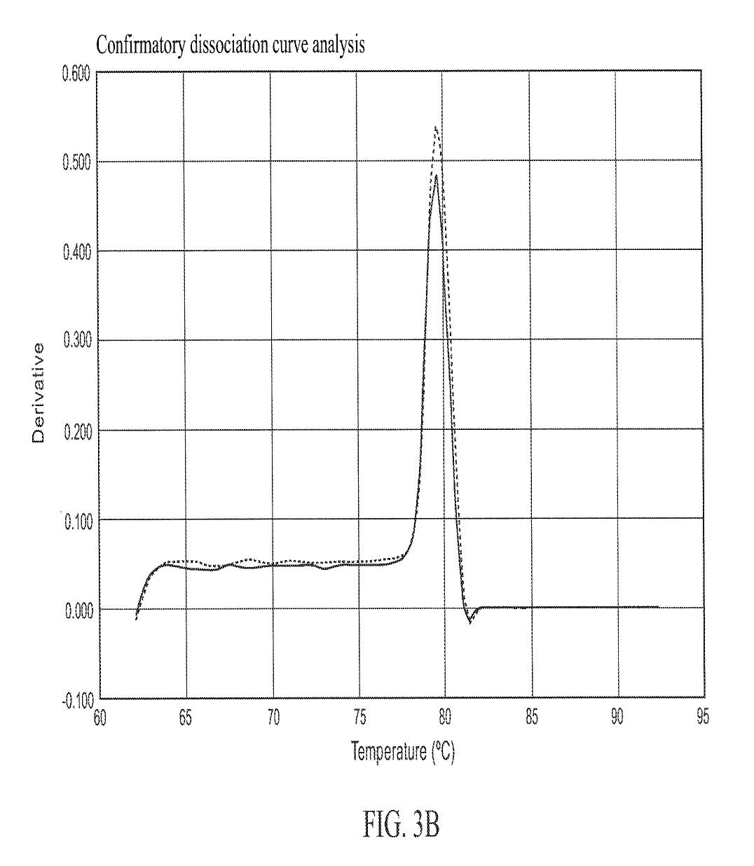

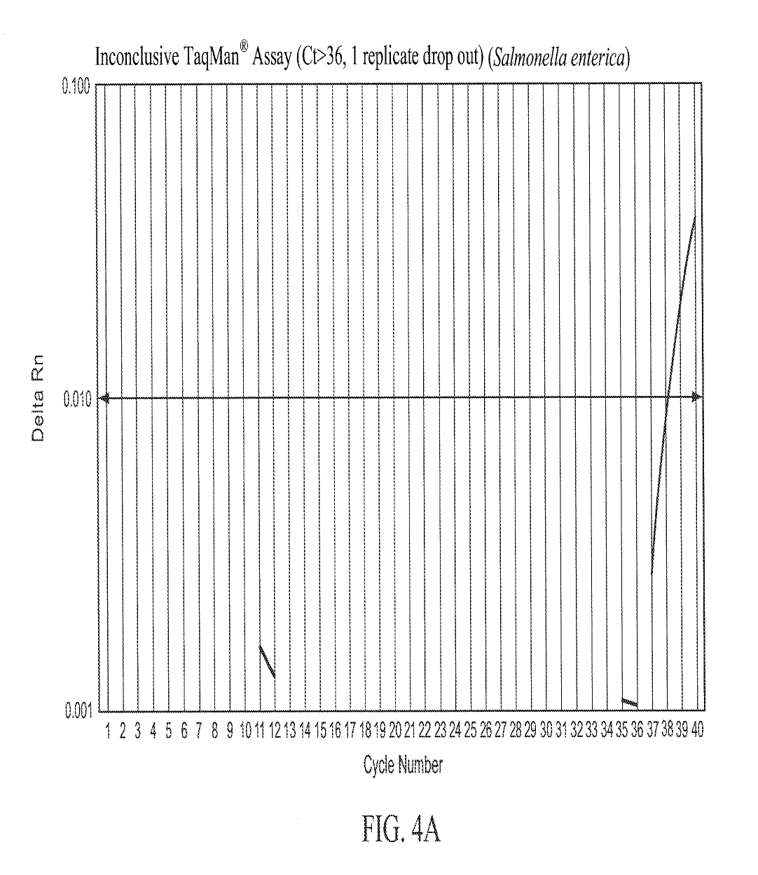

The TaqMelt™ assay can also be used to confirm the results of a “borderline” or an inconclusive TaqMan® assay by following that assay with a dissociation curve analysis (FIG. 3). The assay parameters utilized were the same as those described above (e.g., relating to FIG. 2) except that the amount of S. aureus DNA was ˜0.3 pg (˜50 copies). FIG. 3, panel A illustrates a TaqMan® assay exhibiting Cₜ values (VIC™ channel) of 35.5 (replicate 1) and 36.3 (replicate 2), suggesting borderline results of the assay with Cₜ threshold of 36. FIG. 3, panel B illustrates that melting curve analysis which revealed the presence of amplicon with expected Tₘ of ˜79.6° C. in both replicates. Thus, the TaqMelt™ assay results confirmed that the sample was “positive” for the assayed target.

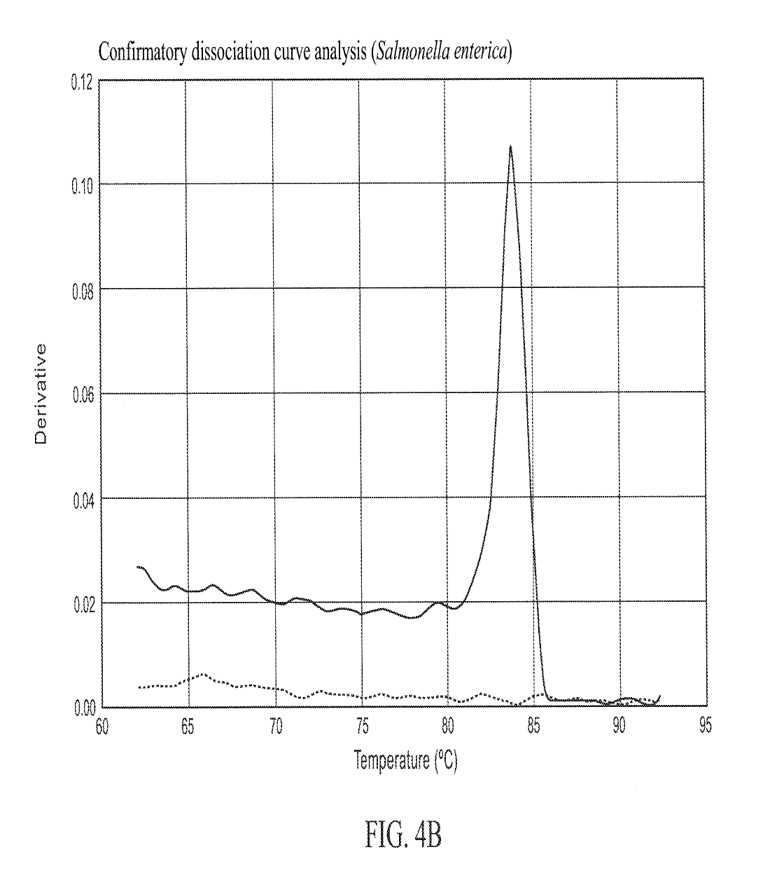

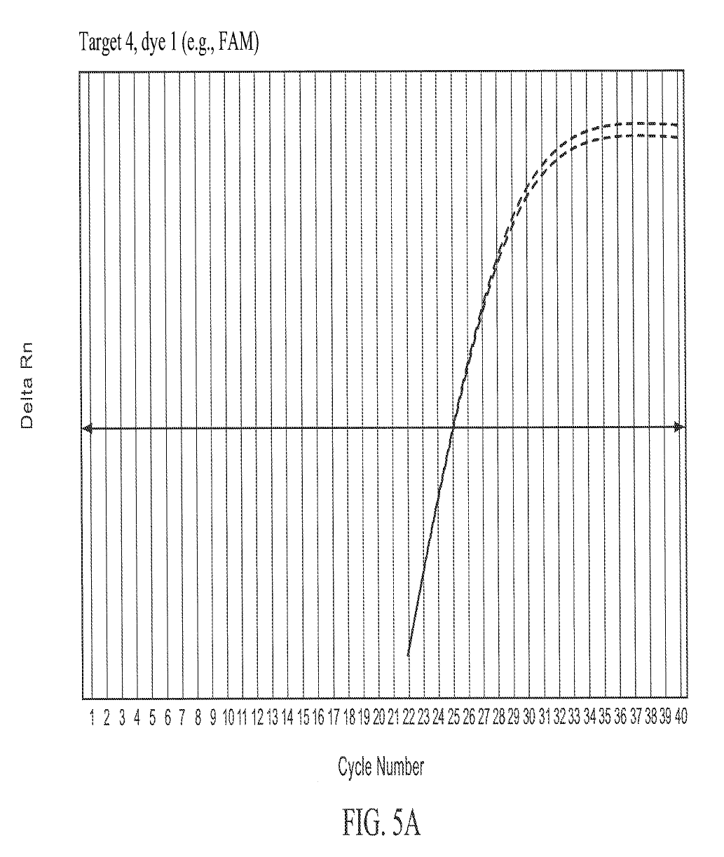

FIG. 4 presents another exemplary confirmation of an “inconclusive” TaqMan® assay using a dissociation curve analysis. The assay parameters were the same as those described above (e.g., relating to FIG. 2) except that the amount of Salmonella enterica DNA in reaction was ˜25 fg (˜5 copies). FIG. 4, panel A, illustrates an assay in which the TaqMan® assay Cₜ values (VIC™ channel) were 38.0 (replicate 1) and >40 (replicate 2), suggesting inconclusive results. FIG. 4, panel B presents a confirmatory melting curve analysis that revealed the presence of amplicon with expected Tₘ of ˜83.9° C. in replicate 1. Thus, the TaqMelt™ assay results confirmed that the sample was actually “positive” for the Salmonella enterica.

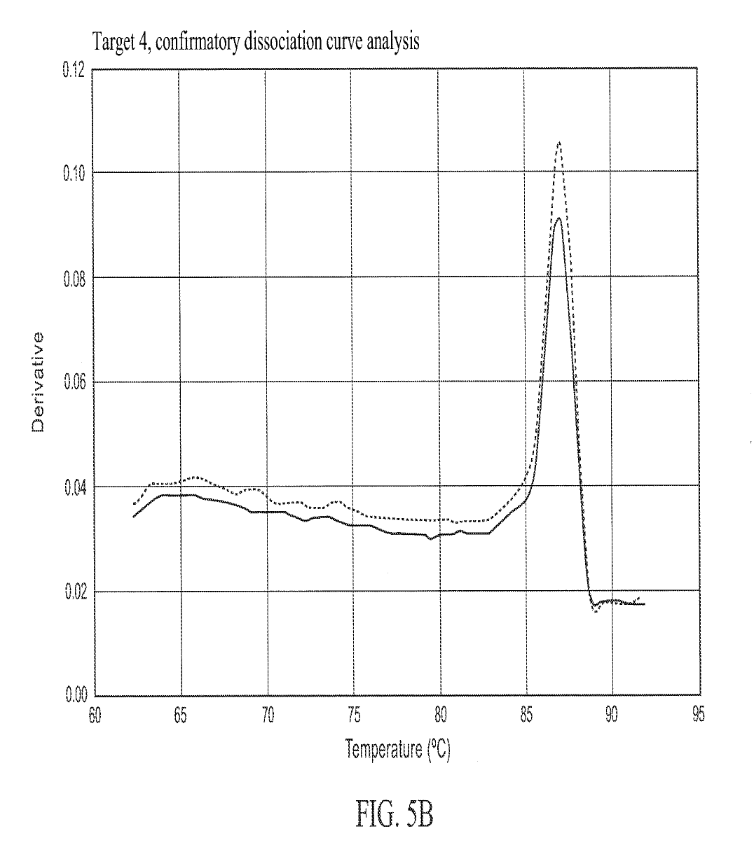

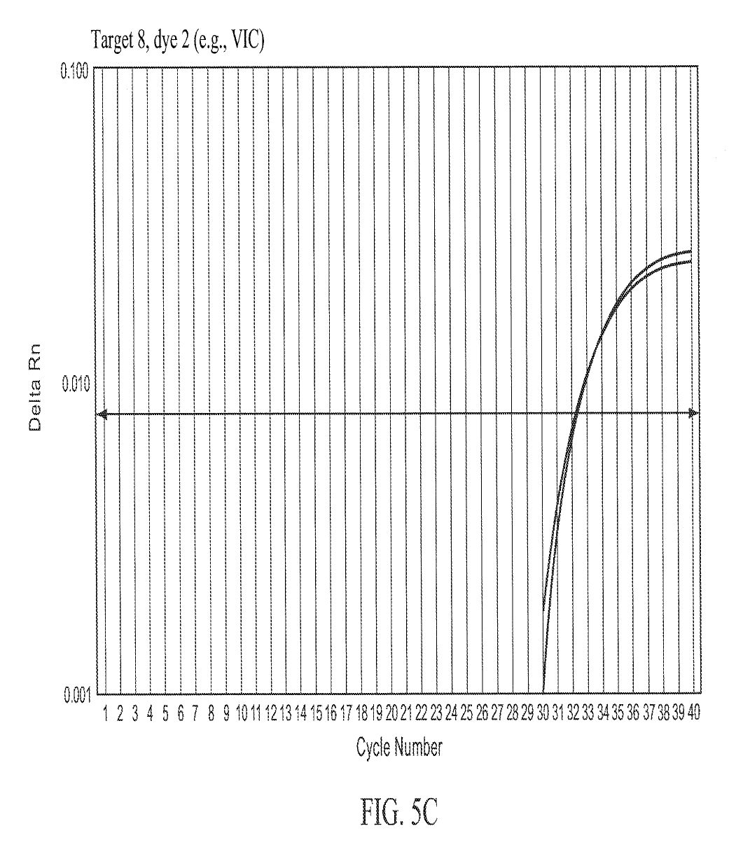

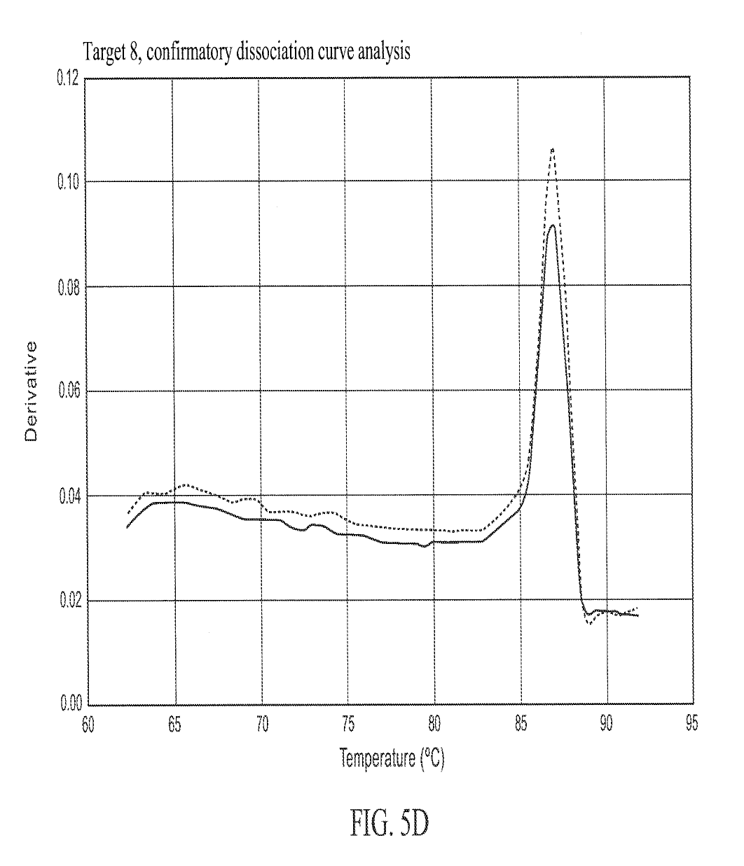

The results of a multiplexed TaqMelt™ assay (e.g., TaqMan® assay followed by dissociation curve analysis) are presented in FIG. 5. The assay parameters were essentially the same as described above (e.g., relating to FIG. 2). Panels A and B (FIG. 5) illustrate the Cₜ analysis using detectable label 1 (FAM™) and the Tₘ analysis for “Target 4”. Panels C and D show the Cₜ analysis using detectable label 2 (VIC™) and the Tₘ analysis for “Target 8” as in Table 1.

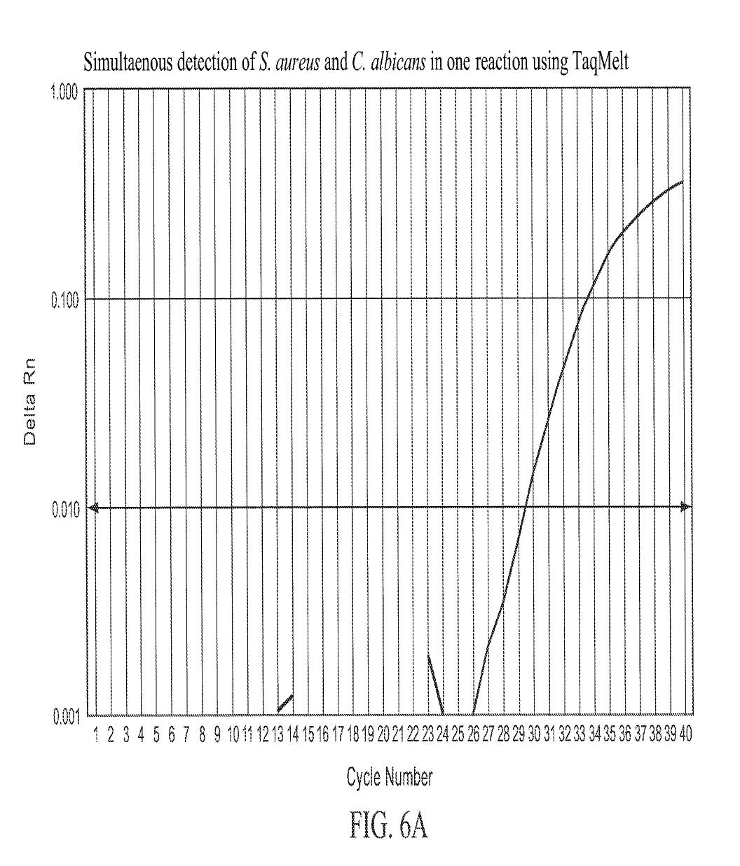

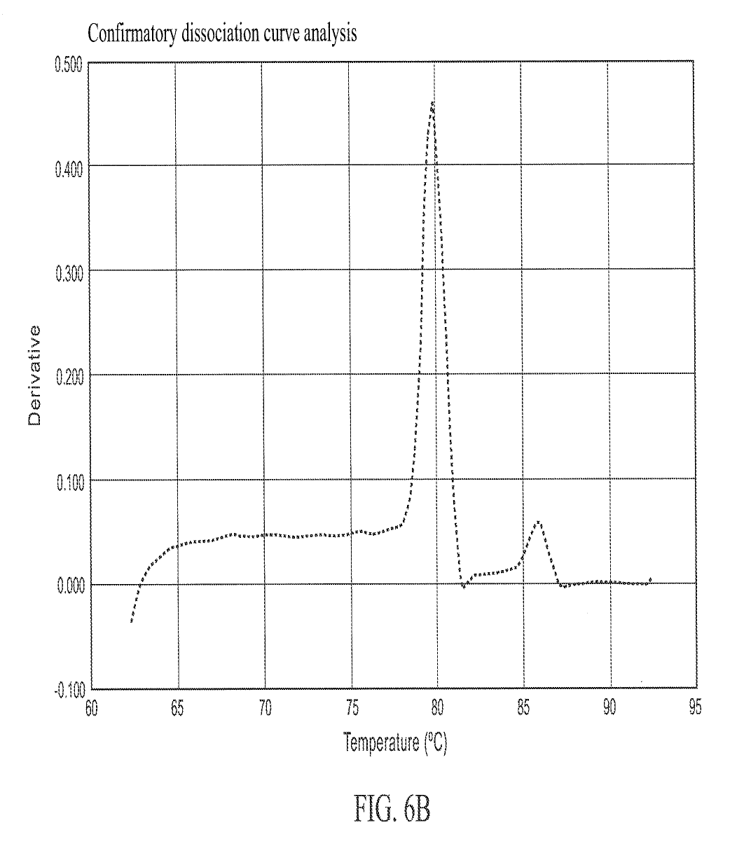

The results of a second exemplary multiplex TaqMelt™ assay (e.g., TaqMan® assay followed by dissociation curve analysis) are illustrated by FIG. 6. The assay parameters were essentially the same as described above (e.g., relating to FIG. 2). In panel A (FIG. 6), the amplification curve was generated for the mixture of S. aureus (˜1000 cfu-s) and C. albicans (˜10 cfu-s) in duplex PCR format. Sequence-specific TaqMan® probes for both targets were labeled with HEX™ dye. Panel B (FIG. 6) presents the melting curve analysis which revealed the presence of two melting peaks with Tₘ-s corresponding to both S. aureus (79.6° C.) and C. albicans (85.9° C.) amplicons. Thus, the TaqMelt™ assay results confirmed that the sample was “positive” for both targets.

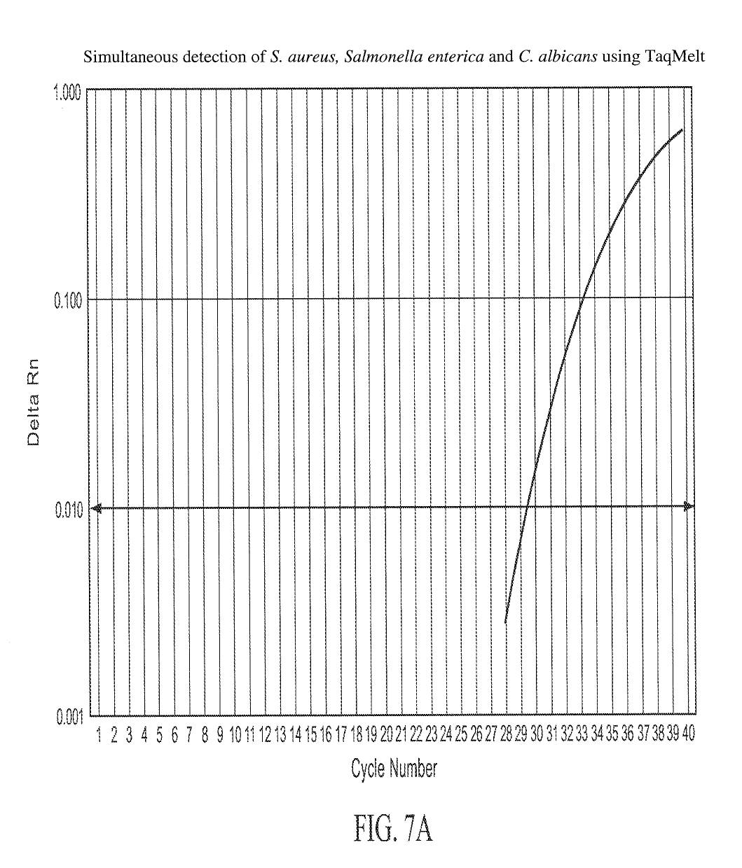

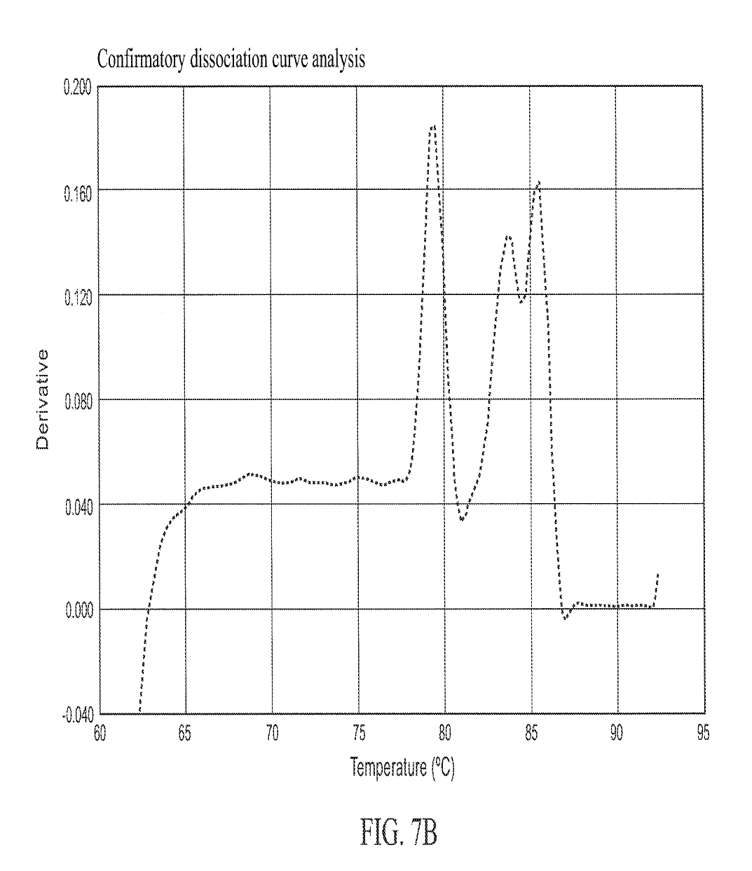

The results of a third exemplary multiplex TaqMelt™ assay (e.g., TaqMan® assay followed by dissociation curve analysis) are shown in FIG. 7. The assay parameters were essentially the same as described above (e.g., relating to FIG. 2). This exemplary assay displays the results using a sample containing S. aureus (˜100 cfu-s), Salmonella enterica (˜10,000 cfu-s) and C. albicans (˜100 cfu-s).

All references cited within this disclosure are hereby incorporated by reference in their entirety. Certain embodiments are further described in the following examples. These embodiments are provided as examples only and are not intended to limit the scope of the claims in any way.

1. A method for detecting at least one target polynucleotide in a biological sample, the method comprising:
a. amplifying a nucleic acid corresponding to a target polynucleotide using at least one primer capable of hybridizing to said target polynucleotide and an oligonucleotide probe capable of hybridizing to said target polynucleotide 3′ relative to said primer,
said oligonucleotide probe having a detectable label capable of being liberated during amplification,
the amplification occurring in the presence of an intercalating dye such that the amplified nucleic acid comprises an intercalating dye;
b. monitoring amplification by detecting the detectable label; and,
c. subsequent to amplification, measuring the melting temperature (Tₘ) of the amplified nucleic acid by monitoring the release of the intercalating dye therefrom.

2. The method of claim 1 wherein the nucleic acid is amplified using a polymerase.

3. The method of claim 2 wherein polymerase comprises 5′ nuclease activity that liberates the oligonucleotide probe.

4. The method of claim 1 wherein the liberating comprises displacement of the oligonucleotide probe by the polymerase.

5. The method of claim 3 wherein the oligonucleotide probe comprises protein nucleic acid (PNA).

6. The method of claim 1 wherein the oligonucleotide probe comprises a quencher molecule and a detectable label and amplification separates the detectable label from the quencher molecule such that the detectable label is not quenched.

7. The method of claim 6 wherein the detectable label is separated from the quencher molecule by a polymerase.

8. The method of claim 7 wherein the polymerase has 5′ nuclease activity.

9. The method of claim 1 comprising at least two oligonucleotide probes, each comprising a different detectable label detectable only when in close proximity to one another.

10. The method of claim 9 wherein the oligonucleotide probes are non-overlapping.

11. The method of claim 1 wherein the detectable label is detected at a temperature other than that used to detect the intercalating dye.

12. The method of claim 11 wherein the amplification reaction of (b) includes a denaturation reaction during which the detectable label is detected.

13. The method of claim 12 wherein the intercalating dye is not detected.

14. The method of claim 1 wherein the target polynucleotide is a double-stranded deoxyribonucleic acid molecule.

15. The method of claim 1 comprising performing nucleic acid amplification on both strands of the target polynucleotide, the intercalating dye being incorporated into both strands.

16. The method of claim 1 wherein multiple probes species are used, each probe species corresponding to a different target polynucleotide.

17. The method of claim 16 comprising different detectable labels on at least two of said probe species.

18. The method of claim 17 comprising different detectable labels on each of said probe species.

19. The method of claim 1 wherein:
an amplified nucleic acid is produced for each of at least two different target polynucleotides, and
at least one of the detectable label on the probe or the Tₘ of the amplified nucleic acid of each target polynucleotide is different from that of any other target polynucleotide.

20. The method of claim 1 wherein the detectable label is selected from the group consisting of a fluoroscein, 5-carboxy-2,7-dichlorofluorescein, 5-carboxyfluorescein (5-FAM), 5-hydroxy tryptamine (HAT), 6-JOE, 6-carboxyfluorescein (6-FAM), FITC, an Alexa fluor, Alexa 350, Alexa 405, Alexa 430, Alexa 488, Alexa 500, Alexa 514, Alexa 532, Alexa 546, Alexa 555, Alexa 568, Alexa 594, Alexa 610, Alexa 633, Alexa 635, Alexa 647, Alexa 660, Alexa 680, Alexa 700, Alexa 750, a BODIPY fluorophore, BODIPY 492/515, BODIPY 493/503, BODIPY 500/510, BODIPY 505/515, BODIPY 530/550, BODIPY 542/563, BODIPY 558/568, BODIPY 564/570, BODIPY 576/589, BODIPY 581/591, BODIPY 630/650-X, BODIPY 650/665-X, BODIPY 665/676, BODIPY FL, BODIPY FL ATP, BODIPY FI-Ceramide, BODIPY R6G SE, BODIPY TMR, BODIPY TMR-X conjugate, BODIPY TMR-X, BODIPY SE, BODIPY TR, BODIPY TR ATP, BODIPY TR-X SE, a coumarin, 7-amino-4-methylcoumarin, AMC, AMCA, AMCA-S, AMCA-X, ABQ, CPM methylcoumarin, coumarin phalloidin, hydroxycoumarin, CMFDA, methoxycoumarin, calcein, calcein AM, calcein blue, a calcium dye, calcium crimson, calcium green, calcium orange, calcofluor white, Cascade Blue, Cascade Yellow; a Cy™ dye, Cy™ 3, Cy™ 3.18, Cy™ 3.5, Cy™ 5, Cy™ 5.18, Cy™ 5.5, Cy™ 7, cyan GFP, cyclic AMP Fluorosensor (FiCRhR), green fluorescent protein, enhanced green fluorescent protein (EGFP), blue fluorescent protein, enhanced blue fluorescent protein (EBFP), enhanced green fluorescent protein 2 (EBFP2), Azurite, mKalama1, cyan fluorescent protein, enhanced cyan fluorescent protein (ECFP), Cerulean, CyPet, yellow fluorescent protein (YFP) Citrine YFP, Venus YFP, YPet YFP, a LysoTracker, a LysoSensor, LysoTracker Blue DND-22, LysoTracker Blue-White DPX, LysoTracker Yellow HCK-123, LysoTracker Green DND-26, LysoTracker Red DND-99, LysoSensor Blue DND-167, LysoSensor Green DND-189, LysoSensor Green DND-153, LysoSensor Yellow/Blue DND-160, LysoSensor Yellow/Blue 10,000 MW dextran, Oregon Green, Oregon Green 488, Oregon Green 488-X, Oregon Green 500, Oregon Green 514, rhodamine, rhodamine 110, rhodamine 123, rhodamine B, rhodamine B 200, rhodamine BB, rhodamine BG, rhodamine B extra, 5-carboxytetramethylrhodamine (5-TAMRA), 5 GLD, 6-Carboxyrhodamine 6G, Lissamine, Lissamine Rhodamine B, Phallicidine, Phalloidine, Red, Rhod-2, carboxy-X-rhodamine (5-ROX), Sulphorhodamine B can C, Sulphorhodamine G Extra, Tetramethylrhodamine (TRITC), WT), Texas Red, Texas Red-X, and VIC™.

21. The method of claim 20 wherein at least two detectable labels are used, the labels being a FRET donor/acceptor pair selected from the group consisting of fluorescein and tetramethylrhodamine, IAEDANS and fluoroscein, EDANS and dabcyl, BODIPY FL and BODIPY FL, and fluorescein QSY7 and fluorescein QSY9.

22. The method of claim 1 wherein the intercalating dye is selected from the group consisting of an acridine, acridine orange, acriflavine, actinomycin D, anthramycin, BOBO™-1, BOBO™-3, BO-PRO™-1, cbromomycin, DAPI, daunomycin, distamycin, distamycin D, ellipticine, an ethidium salt, ethidium bromide, fluorcoumanin, a fluorescent intercalator, GelStar®, Hoechst 33258, Hoechst 33342, homidium, JO-PRO™-1, LO-PRO™-1, mepacrine, mithramycin, an NED dye, netropsin, 4′6-diamidino-α-phenylindole, proflavine, POPO™-1, POPO™-3, PO-PRO™-1, propidium iodide, ruthenium polypyridyls, S5, SYBR® Gold, SYBR® Green I, SYBR® Green II, SYTOX blue, SYTOX green, SYTO® 43, SYTO® 44, SYTO® 45, SYTOX® Blue, TO-PRO®-1, SYTO® 11, SYTO® 13, SYTO® 15, SYTO® 16, SYTO® 20, SYTO® 23, thiazole orange, TOTO™-3, YO-PRO®-1, and YOYO®-3.

23. The method of claim 1 wherein the target polynucleotide is derived from an organism selected from the group consisting of L. monocytogenes, S. aureus, C. jejuni, V. cholerae, E. coli O157:H7, Salmonella spp., S. bongori, E. sakazakii, V. parahaemolyticus, V. vulnificus, C. albicans, A. niger, and P. aeruginosa.

24-25. (canceled)

26. The method of claim 1 wherein the target polynucleotide is derived from a cancerous cell.
